(19) United States
(12) Patent Application Publication
(10) Pub. No.: US 2010/0029728 A1
(43) Pub. Date: Feb. 4, 2010

(54) PHOSPHODIESTERASE INHIBITORS

(75) Inventors: Sarala Balachandran, Mumbai (IN); Nidhi Gupta, New Delhi (IN); Vinayak Vasantrao Khairnar, Nashik (IN); Sonali Rudra, Gurgaon (IN); Venkata P. Palle, Aundh Pune (IN); Sunanda G. Dastidar, New Delhi (IN)

Correspondence Address: Ranbaxy Inc., Intellectual Property Department, 600 College Road East, PRINCETON, NJ, 08540, US

(73) Assignee: RANBAXY LABORATORIES LIMITED, GURGAON HARYANA (IN)

(21) Appl. No.: 12/442,336

(22) PCT Filed: Sep. 22, 2007
(86) PCT No.: PCT/IB07/53855

§ 371 Date: Sep. 14, 2009

(30) Foreign Application Priority Data
Sep. 22, 2006 (IN) 2099/DEL/2006

(51) Int. Cl. A61K 31/4245; C07D 413/04; A61P 29/00; A61P 25/00; A61P 11/06; A61P 11/08; A61P 11/00; A61P 17/06; A61P 27/02; A61P 19/02

(52) U.S. Cl. 514/364; 548/143

(57) ABSTRACT

The present invention relates to phosphodiesterase (PDE) type IV selective inhibitors. Compounds disclosed herein can be useful in the treatment of CNS diseases, AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases especially in humans. Processes for the preparation of disclosed compounds, pharmaceutical compositions containing the disclosed compounds, and their use as PDE type IV selective inhibitors, are provided.

### FIELD OF THE INVENTION

The present invention relates to phosphodiesterase (PDE) type IV selective inhibitors. Compounds disclosed herein can be useful in the treatment of CNS diseases, AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), cosinophilic granuloma, allergic conjunctivitis, osteoarthritis, ulcerative colitis and other inflammatory diseases especially in humans. Processes for the preparation of disclosed compounds, pharmaceutical compositions containing the disclosed compounds, and their use as PDE type IV selective inhibitors, are provided.

### BACKGROUND OF THE INVENTION

It is known that cyclic adenosine-3′,5′-monophosphate (cAMP) exhibits an important role of acting as an intracellular secondary messenger (E. W. Sutherland, and T. W. Roll, Pharmacol. Rev, 1960,12, 265). Its intracellular hydrolysis to adenosine 5′-monophosphate (AMP) causes a number of inflammatory conditions which are not limited to psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis. PDE4 inhibitors are designed to inhibit the activity of PDE4, the enzyme which breaks down neuronal cAMP. Studies have shown that administering PDE4 inhibitors can have a restorative effect on memory loss in animal models, including those of Alzheimer's disease (Expert Opin. Ther. Targets (2005) 9(6):1283-1305; Drug Discovery today, vol. 10, number 22, November 2005). The most important role in the control of cAMP (as well as of cGMP) level is played by cyclic nucleotide phosphodiesterases (PDE) which represent a biochemically and functionally highly variable super family of enzymes; eleven distinct families with more than 25 gene products are currently recognized. Although PIE I, PDE II, PDE III, PDE IV, and PDE VII all use cAMP as a substrate, only PDE IV and PDE VII are highly selective for hydrolysis of cAMP. Inhibitors of PDE, particularly the PDE IV inhibitors, such as rolipram or Ro-1724 are therefore known as cAMP-enhancers. Immune cells contain type IV and type III PDE, the PDE IV type being prevalent in human mononuclear cells. Thus the inhibition of phosphodiesterase type IV has been a target for modulation and, accordingly, for therapeutic intervention in a range of disease processes.

The initial observation that xanthine derivatives, theophylline and caffeine inhibit the hydrolysis of cAMP led to the discovery of the required hydrolytic activity in the cyclic nucleotide phosphodiesterase (PDE) enzymes. Distinct classes of PDE's have been recognized (J. A. Beavo and D. H. Reifsnyder, TIPS, 1990,11,150), and their selective inhibition has led to improved drug therapy (C. D. Nicholus, R. A. Challiss and M, Shahid, TIPS, 1991, 12, 19). Thus it was recognized that inhibition of PDE IV could lead to inhibition of inflammatory mediator release (M. W. Verghese et. al, J. Mol. Cell. Cardiol. 1989, 12 (Suppl. II), S 61) and airway smooth muscle relaxation.

U.S. Pat. No. 5,686,434 discloses 3-aryl-2-isoxazolines as anti-inflammatory agents. U.S. Pat. Nos. 6,114,367 and 5,869,511 disclose isoxazoline compounds as inhibitors of TNF release. WO 95/14681 discloses a series of isoxazoline compounds as anti-inflammatory agents. WO 02/100332 discloses isoxazoline compounds having macrophage inhibitory factor (MIF) antagonist activity.

### SUMMARY OF THE INVENTION

The present invention provides phosphodiesterase inhibitors, which can be used for the treatment of CNS diseases, AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases, and the processes for the synthesis of these compounds.

Pharmaceutically acceptable salts, pharmaceutically acceptable solvates, enantiomers, diastereomers or N-oxides of these compounds having the same type of activity are also provided.

Pharmaceutical compositions containing the compounds, which may also contain pharmaceutically acceptable carriers or diluents, can be used for the treatment of CNS diseases, AIDS, asthma, arthritis, bronchitis, chronic obstructive pulmonary disease (COPD), psoriasis, allergic rhinitis, shock, atopic dermatitis, Crohn's disease, adult respiratory distress syndrome, eosinophilic granuloma, allergic conjunctivitis, osteoarthritis, ulcerative colitis and other inflammatory diseases.

Other aspects will be set forth in the accompanying description which follows and in part will be apparent from the description or may be learnt by the practice of the invention.

In accordance with one aspect, there are provided compounds having the structure of Formula I:

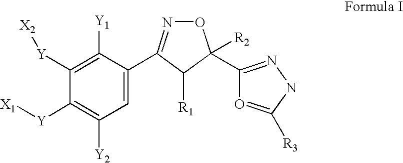

their pharmaceutically acceptable salts, pharmaceutically acceptable solvates, enantiomers, diastereomers or N-oxides, wherein

R₁, R₂ and R₃ can be independently selected from hydrogen or alkyl;

X₁ and X₂ can be independently selected from hydrogen, alkyl, alkenyl, alkynyl, cycloalkyl, aryl, aralkyl, heteroaryl, heterocyclyl, (heteroaryl)alkyl, or (heterocyclyl)alkyl;

Y can represent an oxygen atom, a sulphur atom or NR (wherein R can be selected from hydrogen, alkyl, alkenyl, alkynyl, un(saturated) cycloalkyl, aryl, aralkyl, heteroaryl, heterocyclyl, (heteroaryl)alkyl, or (heterocyclyl)alkyl);

Y₁ and Y₂ can be independently selected from hydrogen, alkyl, nitro, cyano, halogen, OR (wherein R can be the same as defined earlier), SR (wherein R can be the same as defined earlier); NHR (wherein R can be the same as defined earlier), COOR′ or COR′ (wherein R′ can be hydrogen, alkyl, alkenyl, alkynyl, (un)saturated cycloalkyl, aryl, aralkyl, heterocyclyl, (heterocyclyl)alkyl, or (heteroaryl)alkyl);

Further, Y₁ and X₂, X₁ and Y₂, X₁ and X₂ may together form a cyclic ring fused with the ring A containing 3-5 carbon atoms within the ring and having 1-3 heteroatoms selected from N, O or S.

The following definitions apply to terms as used herein:

The term “alkyl,” unless otherwise specified, refers to a monoradical branched or unbranched saturated hydrocarbon chain having from 1 to 20 carbon atoms. This term can be exemplified by groups such as methyl, ethyl, n-propyl, iso-propyl, n-butyl, iso-butyl, sec-butyl, t-butyl, n-pentyl, isopentyl, neopentyl, n-hexyl, n-decyl, tetradecyl, and the like. Alkyl groups may be substituted further with one or more substituents selected from alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, aryl, heterocyclyl, heteroaryl, arylthio, thiol, alkylthio, aryloxy, nitro, aminosulfonyl, aminocarbonylamino, —NHC(═O)R_{f}, —NR_{f}R_{q}, —C(═O)NH₂, —COOR″ (wherein R″ is selected from alkyl, cycloalkyl, aryl, aralkyl, heteroaryl, heterocyclyl, (heteroaryl)alkyl or (heterocyclyl)alkyl), —C(═O)NR_{f}R_{q}, —NHC(═O)NR_{f}R_{q}, —C(═O)heteroaryl, C(═O)heterocyclyl, —O—C(═O)NR_{f}R_{q} {wherein R_{f} and R_{q} are independently selected from alkyl, alkenyl, cycloalkyl, cycloalkenyl, aryl, aralkyl, heterocyclyl, heteroaryl, (heterocyclyl)alkyl, (heteroaryl)alkyl}, nitro, or —SO₂R₆ (wherein R₆ is alkyl, alkenyl, alkynyl, cycloalkyl, aralkyl, aryl, heterocyclyl, heteroaryl, (heteroaryl)alkyl or (heterocyclyl)alkyl). Unless otherwise constrained by the definition, alkyl substituents may be further substituted by 1-3 substituents selected from alkyl, carboxy, —NR_{f}R_{q}, —C(═O)NR_{f}R_{q}, —OC(═O)NR_{f}R_{q}, —NHC(═O)NR_{f}R_{q} (wherein R_{f} and R_{q} are the same as defined earlier), hydroxy, alkoxy, halogen, CF₃, cyano, and —SO₂R₆, (wherein R₆ is the same as defined earlier); or an alkyl group also may be interrupted by 1-5 atoms of groups independently selected from oxygen, sulfur or —NRₐ— {wherein Rₐ is selected from hydrogen, alkyl, cycloalkyl, alkenyl, cycloalkenyl, alkynyl, aryl, acyl, aralkyl, —C(═O)OR_{f} (wherein R_{f} is the same as defined earlier), SO₂R₆ (where R₆ is as defined earlier), or —C(═O)NR_{f}R_{q} (wherein R_{f} and R_{q} are as defined earlier)}. Unless otherwise constrained by the definition, all substituents may be substituted further by 1-3 substituents selected from alkyl, carboxy, —NR_{f}R_{q}, —C(═O)NR_{f}R_{q}, —O—C(═O)NR_{f}R_{q} (wherein R_{f} and R_{q} are the same as defined earlier) hydroxy, alkoxy, halogen, CF₃, cyano, and ‘3SO₂R₆ (where R₆ is same as defined earlier); or an alkyl group as defined above that has both substituents as defined above and is also interrupted by 1-5 atoms or groups as defined above.

The term “alkenyl,” unless otherwise specified, refers to a monoradical of a branched or unbranched unsaturated hydrocarbon group having from 2 to 20 carbon atoms with cis, trans, or geminal geometry. In the event that alkenyl is attached to a heteroatom, the double bond cannot be alpha to the heteroatom. Alkenyl groups may be substituted further with one or more substituents selected from alkyl, alkynyl, alkoxy, cycloalkyl, cycloalkenyl, acyl, acylamino, acyloxy, —NHC (═O)R_{f}, —NR_{f}R_{q}, —C(═O)NR_{f}R_{q}, —NHC(═O)NR_{f}R_{q}, —O—C(═O)NR_{f}R_{q} (wherein R_{f} and R_{q} are the same as defined earlier), alkoxycarbonylamino, azido, cyano, halogen, hydroxy, oxo, thiocarbonyl, carboxy, arylthio, thiol, alkylthio, aryl, aralkyl, aryloxy, heterocyclyl, heteroaryl, (heterocyclyl)alkyl, (heteroaryl)alkyl, aminosulfonyl, aminocarbonylamino, alkoxyamino, nitro, or SO₂R₆ (wherein R₆ is same as defined earlier). Unless otherwise constrained by the definition, alkenyl substituents optionally may be substituted further by 1-3 substituents selected from alkyl, carboxy, hydroxy, alkoxy, halogen, —CF₃, cyano, —NR_{f}R_{q}, —C(═O)NR_{f}R_{q}, —O—C(═O)NR_{f}R_{q} (wherein R_{f} and R_{q} are the same as defined earlier) and —SO₂R₆ (where R₆ is same as defined earlier).

The term “alkynyl,” unless otherwise specified, refers to a monoradical of an unsaturated hydrocarbon, having from 2 to 20 carbon atoms. In the event that alkynyl is attached to a heteroatom, the triple bond cannot be alpha to the heteroatom. Alkynyl groups may be substituted further with one or more substituents selected from alkyl, alkenyl, alkoxy, cycloalkyl, cycloalkenyl, acyl, acylamino, acyloxy, alkoxycarbonylamino, azido, cyano, halogen, hydroxy, oxo, thiocarbonyl, carboxy, arylthio, thiol, alkylthio, aryl, aralkyl, aryloxy, aminosulfonyl, aminocarbonylamino, nitro, heterocyclyl, heteroaryl, (heterocyclyl)alkyl, (heteroaryl)alkyl, —NHC(═O)R_{f}, —NR_{f}R_{q}, —NHC(═O)NR_{f}R_{q}, —C(═O)NR_{f}R_{q}, 13 O—C(═O)NR_{f}R_{q} (wherein R_{f} and R_{q} are the same as defined earlier), or —SO₂R₆ (wherein R₆ is as defined earlier). Unless otherwise constrained by the definition, alkynyl substituents optionally may be substituted further by 1-3 substituents selected from alkyl, carboxy, carboxyalkyl, hydroxy, alkoxy, halogen, CF₃, —NR_{f}R_{q}, —C(═O)NR_{f}R_{q}, —NHC(═O)NR_{f}R_{q}, —C(═O)NR_{f}R_{q} (wherein R_{f} and R_{q} are the same as defined earlier), cyano, or —SO₂R₆ (where R₆ is same as defined earlier).

The term “cycloalkyl,” unless otherwise specified, refers to cyclic alkyl groups of from 3 to 20 carbon atoms having a single cyclic ring or multiple condensed rings, which may optionally contain one or more olefinic bonds, unless otherwise constrained by the definition. Such cycloalkyl groups can include, for example, single ring structures, including cyclopropyl, cyclobutyl, cyclooctyl, cyclopentenyl, and the like, or multiple ring structures, including adamantanyl, and bicyclo[2.2.1]heptane, or cyclic alkyl groups to which is fused an aryl group, for example, indane, and the like. Spiro and fused ring structures can also be included. Cycloalkyl groups may be substituted further with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, arylthio, thiol, alkylthio, aryl, aralkyl, aryloxy, aminosulfonyl, aminocarbonylamino, —NR_{f}R_{q}, —NHC(═O)NR_{f}R_{q}, —NHC(═O)R_{f}, —C(═O)NR_{f}R_{q}, —O—C(═O)NR_{f}R_{q} (wherein R_{f} and R_{q} are the same as defined earlier), nitro, heterocyclyl, heteroaryl, (heterocyclyl)alkyl, (heteroaryl)alkyl, or —SO₂R₆ (wherein R₆ is same as defined earlier). Unless otherwise constrained by the definition, cycloalkyl substituents optionally may be substituted further by 1-3 substituents selected from alkyl, carboxy, hydroxy, alkoxy, halogen, CF₃, —NR_{f}R_{q}, —C(═O)NR_{f}R_{q}, —NHC(═O)NR_{f}R_{q}, —O—C(═O)NR_{f}R_{q} (wherein R_{f} and R_{q} are the same as defined earlier), cyano or —SO₂R₆ (where R₆ is same as defined earlier).

The term “alkoxy” denotes the group O-alkyl, wherein alkyl is the same as defined above.

The term “aryl,” unless otherwise specified, refers to carbocyclic aromatic groups, for example, phenyl, biphenyl or napthyl ring and the like, optionally substituted with 1 to 3 substituents selected from halogen (e.g., F, Cl, Br, I), hydroxy, alkyl, alkenyl, alkynyl, cycloalkyl, alkoxy, acyl, aryloxy, CF₃, cyano, nitro, COORₑ (wherein Rₑ is hydrogen, alkyl, alkenyl, cycloalkyl, aralkyl, (heterocyclyl)alkyl, (heteroaryl)alkyl), NHC(═O)R_{f}, —NR_{f}R_{q}, —C(═O)NR_{f}R_{q}, —NHC(═O)NR_{f}R_{q}, —O—C(═O)NR_{f}R_{q} (wherein R_{f} and R_{q} are the same as defined earlier), —SO₂R₆ (wherein R₆ is same as defined earlier), carboxy, heterocyclyl, heteroaryl, (heterocyclyl)alkyl, (heteroaryl)alkyl or amino carbonyl amino.

The aryl group optionally may be fused with a cycloalkyl group, wherein the cycloalkyl group may optionally contain heteroatoms selected from O, N or S.

The term “aralkyl,” unless otherwise specified, refers to alkyl-aryl linked through an alkyl portion (wherein alkyl is as defined above) and the alkyl portion contains 1-6 carbon atoms and aryl is as defined below. Examples of aralkyl groups include benzyl, ethylphenyl and the like.

The term “aralkenyl,” unless otherwise specified, refers to alkenyl-aryl linked through alkenyl (wherein alkenyl is as defined above) portion and the alkenyl portion contains 1 to 6 carbon atoms and aryl is as defined below.

The term “aryloxy” denotes the group O-aryl, wherein aryl is as defined above.

The term “carboxy,” as defined herein, refers to —C(═O)OH.

The term “heteroaryl,” unless otherwise specified, refers to an aromatic ring structure containing 5 or 6 ring atoms, or a bicyclic aromatic group having from 8 to 10 ring atoms, with one or more heteroatom(s) independently selected from N, O or S optionally substituted with 1 to 4 substituent(s) selected from halogen (e.g., F, Cl, Br, I), hydroxy, alkyl, alkenyl, alkynyl, cycloalkyl, acyl, carboxy, aryl, alkoxy, aralkyl, cyano, nitro, heterocyclyl, heteroaryl, —NR_{f}R_{q}, CH═NOH, —(CH₂)_{w}C(═O)R_{g} {wherein w is an integer from 0-4 and R_{g} is hydrogen, hydroxy, OR_{f}, NR_{f}R_{q}, —NHOR_{z} or —NHOH}, —C(═O)NR_{f}R_{q} and —NHC(═O)NR_{f}R_{q}, —SO₂R₆, —O—C(═O)NR_{f}R_{q}, —O—C(═O)R_{f}, —O—C(═O)OR_{f} (wherein R₆, R_{f} and R_{q} are as defined earlier, and R_{z} is alkyl, cycloalkyl, aryl, heteroaryl, heterocyclyl, (heteroaryl)alkyl or (heterocyclyl)alkyl). Unless otherwise constrained by the definition, the substituents are attached to a ring atom, i.e., carbon or heteroatom in the ring. Examples of heteroaryl groups include oxazolyl, imidazolyl, pyrrolyl, 1,2,3-triazolyl, 1,2,4-triazolyl, tetrazolyl, thiazolyl, oxadiazolyl, benzoimidazolyl, thiadiazolyl, pyridinyl, pyridazinyl, pyrimidinyl, pyrazinyl, thienyl, isoxazolyl, triazinyl, furanyl, benzofuranyl, indolyl, benzothiazolyl, or benzoxazolyl, and the like.

The term ‘heterocyclyl,’ unless otherwise specified, refers to a non-aromatic monocyclic or bicyclic cycloalkyl group having 3 to 10 atoms wherein 1 to 4 carbon atoms in a ring are replaced by heteroatoms selected from O, S or N, and their oxidized forms, and optionally are benzofused or fused heteroaryl having 5-6 ring members and/or optionally are substituted, wherein the substituents are selected from halogen (e.g., F, Cl, Br, I), hydroxy, alkyl, alkenyl, alkynyl, cycloalkyl, acyl, aryl, alkoxy, alkaryl, cyano, nitro, oxo, carboxy, heterocyclyl, heteroaryl, —O—C(═O)R_{f}, —O—C(═O)OR_{f}, —C(═O)NR_{f}R_{q}, SO₂R₆, —O—C(═O)NR_{f}R_{q}, —NHC(═O)NR_{f}R_{q}, —NR_{f}R_{q} (wherein R₆, R_{f} and R_{q} are as defined earlier) or guanidine. Heterocyclyl can optionally include rings having one or more double bonds. Unless otherwise constrained by the definition, the substituents are attached to the ring atom, i.e., carbon or heteroatom in the ring. Also, unless otherwise constrained by the definition, the heterocyclyl ring optionally may contain one or more olefinic bond(s). Examples of heterocyclyl groups include oxazolidinyl, tetrahydrofuranyl, dihydrofuranyl, dihydropyridinyl, dihydroisoxazolyl, dihydrobenzofuryl, azabicyclohexyl, dihydroindolyl, pyridinyl, isoindole 1,3-dione, piperidinyl, morpholinyl or piperazinyl.

“(Heteroaryl)alkyl” refers to alkyl-heteroaryl group linked through alkyl portion, wherein the alkyl and heteroaryl are as defined earlier.

“(Heterocyclyl)alkyl” refers to alkyl-heterocyclyl group linked through alkyl portion, wherein the alkyl and heterocyclyl are as defined earlier.

“Acyl” refers to —C(═O)R′, wherein R′ is selected from hydrogen, alkyl, alkenyl, alkynyl, (un)saturated cycloalkyl, aryl, aralkyl, heterocyclyl, (heterocyclyl)alkyl, or (heteroaryl)alkyl.

“Alkylcarbonyl” refers to —C(═O)R″, wherein R″ is selected from alkyl, cycloalkyl, aryl, aralkyl, heteroaryl, heterocyclyl, (heteroaryl)alkyl or (heterocyclyl)alkyl.

“Alkylcarboxy” refers to —O—C(═O)R″, wherein R″ is selected from alkyl, cycloalkyl, aryl, aralkyl, heteroaryl, heterocyclyl, (heteroaryl)alkyl or (heterocyclyl)alkyl.

“Amine,” unless otherwise specified, refers to —NH₂, “Substituted amine,” unless otherwise specified, refers to —N(Rₖ)₂, wherein each Rₖ independently is selected from hydrogen {provided that both Rₖ groups are not hydrogen (defined as “amino”)}, alkyl, alkenyl, alkynyl, aralkyl, cycloalkyl, aryl, heteroaryl, heterocyclyl, (heterocyclyl)alkyl, (heteroaryl)alkyl, acyl, SO₂R₆ (wherein R₆ is as defined above), —C(═O)NR_{f}R_{q}, NHC(═O)NR_{f}R_{q}, or —NHC(═O)OR_{f} (wherein R_{f} and R_{q} are as defined earlier).

“Thiocarbonyl” refers to —C(═S)H. “Substituted thiocarbonyl” refers to —C(═S)R″, wherein R″ is selected from alkyl, cycloalkyl, aryl, aralkyl, heteroaryl, heterocyclyl, (heteroaryl)alkyl or (heterocyclyl)alkyl, amine or substituted amine.

Unless otherwise constrained by the definition, all substituents optionally may be substituted further by 1-3 substituents selected from alkyl, aralkyl, cycloalkyl, aryl, heteroaryl, heterocyclyl, carboxy, carboxyalkyl, hydroxy, alkoxy, halogen, CF₃, cyano, —C(═O)NR_{f}R_{q}, —O(C═O)NR_{f}R_{q} (wherein R_{f} and R_{q} are the same as defined earlier) and —SO₂R₆ (where R₆ is the same as defined earlier).

The term “leaving group” refers to groups that exhibit or potentially exhibit the properties of being labile under the synthetic conditions and also, of being readily separated from synthetic products under defined conditions. Examples of leaving groups include, but are not limited to, halogen (e.g., F, Cl, Br, I), triflates, tosylate, mesylates, alkoxy, thioalkoxy, or hydroxy radicals and the like.

The term “protecting groups” refers to moieties that prevent chemical reaction at a location of a molecule intended to be left unaffected during chemical modification of such molecule. Unless otherwise specified, protecting groups may be used on groups, such as hydroxy, amino, or carboxy. Examples of protecting groups are found in T. W. Greene and P. G. M. Wuts, “Protective Groups in Organic Synthesis”, 2^{nd} Ed., John Wiley and Sons, New York, N.Y., which is incorporated herein by reference. The species of the carboxylic protecting groups, amino protecting groups or hydroxy protecting groups employed are not critical, as long as the derivatised moieties/moiety is/are stable to conditions of subsequent reactions and can be removed without disrupting the remainder of the molecule. Certain “protecting groups” may be formed in situ under the reaction conditions and may be removed when the conditions under which they are formed are modified. Example of such protection is the lithiation of hydroxyl groups under lithiation conditions.

The term “pharmaceutically acceptable salts” refers to derivatives of compounds that can be modified by forming their corresponding acid or base salts. Examples of pharmaceutically acceptable salts include, but are not limited to, mineral or organic acids salts of basic residues (such as amines), or alkali or organic salts of acidic residues (such as carboxylic acids), and the like.

The compounds provided herein can be used for treating CNS diseases, AIDS, asthma, arthritis, bronchitis, chronic obstructive pulmonary disease, psoriasis, allergic rhinitis, shock, atopic dermatitis, Crohn's disease, adult respiratory distress syndrome, eosinophilic granuloma, allergic conjunctivitis, osteoarthritis, ulcerative colitis and other inflammatory diseases.

In accordance with yet another aspect, there are provided processes for the preparation of the compounds as described herein.

### DETAILED DESCRIPTION OF THE INVENTION

The compounds described herein may be prepared by techniques well known in the art and familiar to the average synthetic organic chemist. In addition, the compounds of present invention may be prepared by the following reaction sequences as depicted in schemes I, II, III IV and V.

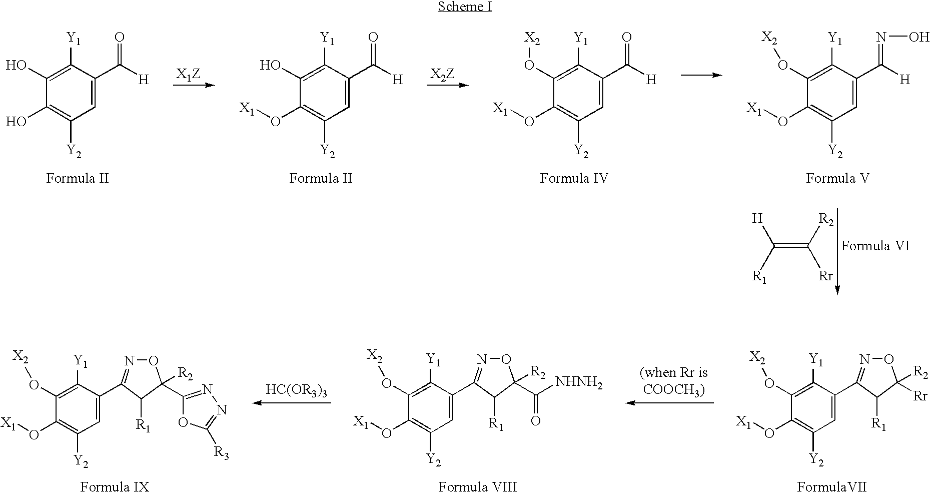

The compounds of Formula IX can be prepared by following Scheme I. Accordingly, reacting a compound of Formula II with a compound of Formula X₁Z (wherein Z is halogen) can give a compound of Formula III [wherein X₁ (except hydrogen), Y₁ and Y₂ are the same as defined earlier], which on reaction with a compound of Formula X₂Z [wherein Z is halogen] can give a compound of Formula IV [wherein X₂ (except hydrogen) is same as defined earlier], which on reaction with hydroxylamine hydrochloride can give a compound of Formula V, which on treatment with a compound of Formula VI can give a compound of Formula VII [wherein R₁ and R₂ are the same as defined earlier and Rr represents COOH, COOCH₃], which (when Rr is COOCH₃) on reaction with hydrazine hydrate can give a compound of Formula VIII, which can finally be reacted with a compound of Formula HC(OR₃)₃ to give a compound of Formula IX [wherein R₃ is the same as defined earlier].

The reaction of a compound of Formula It with a compound of Formula X₁Z to give a compound of Formula III can be carried out in the presence of one or more of phase transfer catalysts, for example, benzyltributyl ammonium chloride, benzyltriethylammonium chloride, benzyltriethylammonium iodide or mixtures thereof.

The reaction of a compound of Formula II with a compound of Formula X₁Z can be carried out in the presence of one or more of inorganic bases, for example, alkali metal hydroxides, for example, sodium hydroxide, potassium hydroxide, lithium hydroxide, alkali metal carbonates, for example, potassium carbonate, cesium carbonate or mixtures thereof.

The reaction of a compound of Formula II with a compound of Formula X₁Z can be carried out in one or more of solvents, for example, tetrahydrofuran, dimethylformamide, dimethylsulphoxide, acetonitrile, dimethylacetamide or mixtures thereof.

The reaction of a compound of Formula III with a compound of Formula X₂Z can be carried out in the presence of one or more of inorganic bases, for example, sodium carbonate, sodium bicarbonate, potassium carbonate, potassium bicarbonate, cesium carbonate or mixtures thereof.

The reaction of a compound of Formula III with a compound of Formula X₂Z to give a compound of Formula IV can be carried out in one or more of solvents, for example, tetrahydrofuran, dimethylformamide, dimethylsulphoxide, acetonitrile, acetone, dimethylacetamide or mixtures thereof.

The reaction of a compound of Formula IV with hydroxylamine hydrochloride to give a compound of Formula V can be carried out in the presence of sodium acetate, potassium acetate, triethylamine or pyridine in one or more of solvents, for example, methanol, ethanol, propanol, n-butanol or mixtures thereof.

The reaction of a compound of Formula V with a compound of Formula VI to give a compound of Formula VII can be carried out in the presence of sodium hypochlorite in one or more of solvents, for example, tetrahydrofuran, dimethylformamide, dimethylsulphoxide, acetonitrile, chloroform, dichloromethane or mixtures thereof.

The reaction of a compound of Formula VII with hydrazine hydrate to give a compound of Formula VIII can be carried out at a temperature ranging, for example, from 120 to 140° C.

The reaction of a compound of Formula VIII with a compound of Formula HC(OR₃)₃ to give a compound of Formula IX can be carried out at a temperature ranging, for example, from 60 to 160° C.

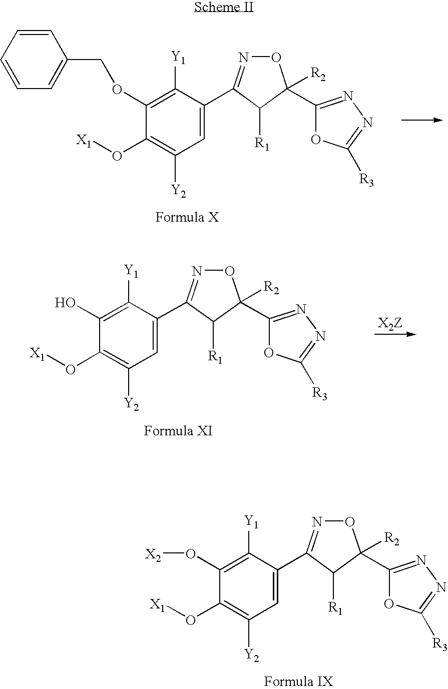

The compounds of Formula IX can also be prepared by following Scheme II. Accordingly, a compound of Formula X on debenzylation can give a compound of Formula XI [wherein X₁, Y₁, Y₂, R₁, R₂ and R₃ are the same as defined earlier], which, finally on reaction with X₂Z [wherein Z is halogen] can give a compound of Formula IX [wherein X₂ (except hydrogen and benzyl) is same as defined earlier].

The debenzylation of a compound of Formula X to give a compound of Formula XI can be carried out by catalytic transfer hydrogenation in the presence of one or more of palladium catalysts or ammonium formate or in the presence of boron tribromide in one or more of solvents, for example, methanol, ethanol, propanol, n-butanol, toluene or mixtures thereof.

The reaction of a compound of Formula XI with a compound of Formula X₂Z to give a compound of Formula IX can be carried out in the presence of one or more of inorganic bases, for example, sodium carbonate, sodium bicarbonate, potassium carbonate, cesium carbonate, potassium bicarbonate or mixtures thereof.

The reaction of a compound of Formula XI with a compound of Formula X₂Z can be carried out in one or more of solvents, for example, tetrahydrofuran, dimethylformamide, dimethylsulphoxide, acetonitrile, acetone, dimethylacetamide or mixtures thereof.

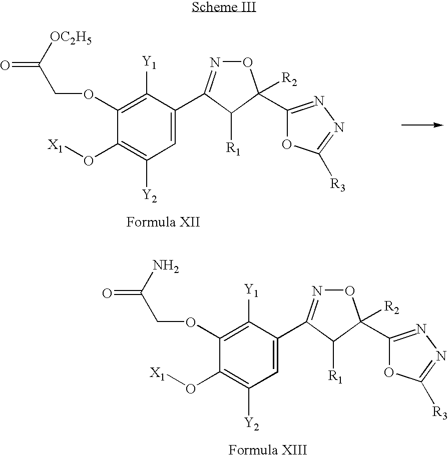

The compounds of Formula XIII can be prepared by following Scheme III. Accordingly, a compound of Formula XII can be amidated to give a compound of Formula XIII [wherein X₁, Y₁, Y₂, R₁, R₂ and R₃ are the same as defined earlier].

The amidation of a compound of Formula XII to give a compound of Formula XIII can be carried out in the presence of methanolic ammonia or an alkylamine.

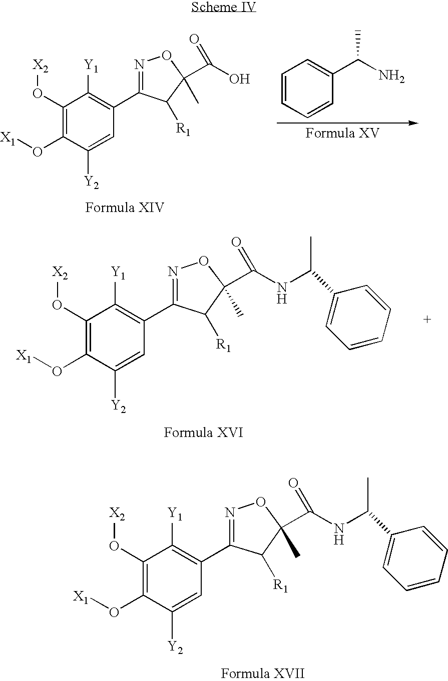

The compounds of Formula XVI and Formula XVII can be prepared by following Scheme IV. Accordingly, a compound of Formula XIV can be reacted with a compound of Formula XV to give a compound of Formula XVI [wherein X₁, X₂, Y₁, Y₂ and R₁ are the same as defined earlier] and a compound of Formula XVII [wherein X₁, X₂, Y₁, Y₂ and R₁ are the same as defined earlier].

The reaction of a compound of Formula XIV with a compound of Formula XV to give a compound of Formula XVI and a compound of Formula XVII can be carried out in the presence of one or more of halogenating agents, for example, thionyl chloride, oxalyl chloride, sulfuryl chloride, phosphorus oxychloride, phosphorus trichloride, phosphorus pentachloride, phosphorus tribromide or mixtures thereof.

The reaction of a compound of Formula XIV with a compound of Formula XV can be carried out in one or more of solvents, for example, benzene, toluene, dichloromethane, chloroform or mixtures thereof.

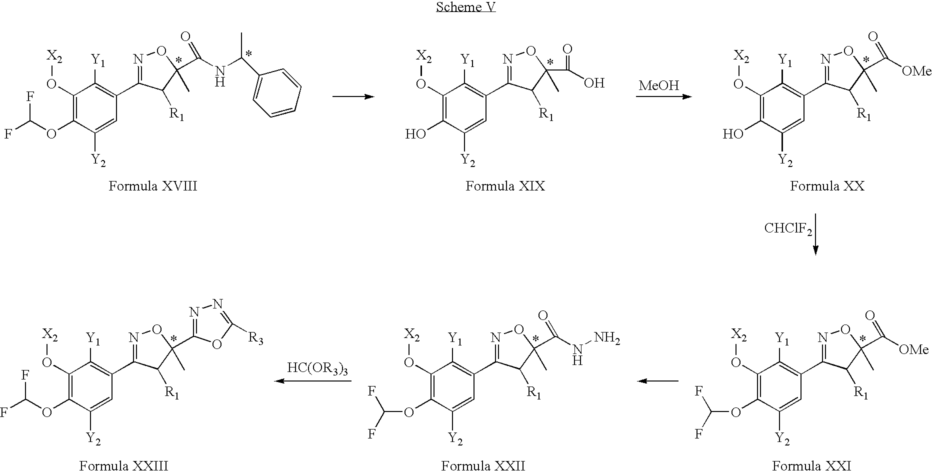

The compounds of Formula XXIII can be prepared by following Scheme V. Accordingly, a compound of Formula XVIII [wherein configuration at stereogenic carbons marked * can be (R) or (S)] on reaction with hydrazine hydrate can give a compound of Formula XIX, which on reaction with methanol can give a compound of Formula XX, which on reaction with Freon gas can give a compound of Formula XXI, which on reaction with hydrazine hydrate can give a compound of Formula XXII, which can, finally, be reacted with a compound of Formula HC(OR₃)₃ to give a compound of Formula XXIII [wherein X₂, Y₁, Y₂, R₁ and R₃ are the same as defined earlier and configuration at stereogenic carbon marked * can be (R) or (S)].

The reaction of a compound of Formula XVIII with hydrazine hydrate to give a compound of Formula XIX can be carried out in the presence of one or more of inorganic bases, for example, potassium hydroxide, sodium hydroxide, lithium hydroxide, cesium hydroxide or mixtures thereof.

The reaction of a compound of Formula XVIII with hydrazine hydrate can be carried out in one or more of solvents, for example, methanol, ethanol, propanol, isopropanol, ethylene glycol or mixtures thereof.

The reaction of a compound of Formula XIX with methanol to give a compound of Formula XX can be carried out in the presence of one or more of mineral acids, for example, sulphuric acid, hydrochloric acid or mixtures thereof.

The reaction of a compound of Formula XX with Freon gas to give a compound of Formula XXI can be carried out in the presence of one or more of phase transfer catalysts, for example, benzyltributylammonium chloride, benzyltriethylammonium chloride, benzyltriethylammonium iodide or mixtures thereof.

The reaction of a compound of Formula XX with Freon gas can be carried out in the presence of one or more of inorganic bases, for example, potassium carbonate, potassium bicarbonate, sodium carbonate, sodium bicarbonate, cesium carbonate or mixtures thereof.

The reaction of a compound of Formula XX with Freon gas can be carried out in one or more of solvents, for example tetrahydrofuran, dimethylformamide, dimethylsulphoxide, acetonitrile, dimethylacetamide or mixtures thereof.

The reaction of a compound of Formula XXI with hydrazine hydrate to give a compound of Formula XXII can be carried out at a temperature ranging, for example, from 120 to 140° C.

The reaction of a compound of Formula XXII with a compound of Formula HC(OR₁₁)₃ to give a compound of Formula XXIII can be carried out at a temperature ranging, for example, from 60 to 160° C.

An illustrative list of compounds of the invention is listed below
2-{3-[3-(Benzyloxy)-4-(difluoromethoxy)phenyl]-5-methyl-4,5-dihydroisoxazol-5-yl}-1,3,4-oxadiazole (compound no. 1),
2-(Difluoromethoxy)-5-[5-methyl-5-(1,3,4-oxadiazol-2-yl)-4,5-dihydroisoxazol-3-yl]phenol (compound no. 2),
Ethyl {2-methoxy-5-[5-methyl-5-(1,3,4-oxadiazol-2-yl)-4,5-dihydroisoxazol-3-yl]phenoxy}acetate (compound no. 3),
2-Methoxy-5-[5-methyl-5-(1,3,4-oxadiazol-2-yl)-4,5-dihydroisoxazol-3-yl]phenol (compound no. 4),
Ethyl {2-(difluoromethoxy)-5-[5-methyl-5-(1,3,4-oxadiazol-2-yl)-4,5-dihydroisoxazol-3-yl]phenoxy}acetate (compound no. 5),
2-{2-Methoxy-5-[5-methyl-5-(1,3,4-oxadiazol-2-yl)-4,5-dihydroisoxazol-3-yl]phenoxy}ethanol (compound no. 6),
4-(2-{2-Methoxy-5-[5-methyl-5-(1,3,4-oxadiazol-2-yl)-4,5-dihydroisoxazol-3-yl]phenoxy}ethyl)morpholine (compound no. 7),
2-{3-[3-(Benzyloxy)-4-methoxyphenyl]-5-methyl-4,5-dihydroisoxazol-5-yl}-1,3,4-oxadiazole (compound no. 8),
2-{2-(Difluoromethoxy)-5-[5-methyl-5-(1,3,4-oxadiazol-2-yl)-4,5-dihydroisoxazol-3-yl]phenoxy}acetamide (compound no. 9),
2-{2-(Difluoromethoxy)-5-[5-methyl-5-(1,3,4-oxadiazol-2-yl)-4,5-dihydroisoxazol-3-yl]phenoxy}ethanol (compound no. 10),
2-{(5S or 5R)-3-[4-(Difluoromethoxy)-3-ethoxyphenyl]-5-methyl-4,5-dihydroisoxazol-5-yl}-1,3,4-oxadiazole (compound no. 11),
2-{(5R or 5S)-3-[4-(Difluoromethoxy)-3-ethoxyphenyl]-5-methyl-4,5-dihydroisoxazol-5-yl}-1,3,4-oxadiazole (compound no. 12),
   pharmaceutically acceptable salts, pharmaceutically acceptable solvates, enantiomers, diastereomers or N-oxides thereof.

The following compounds can be prepared by following the schemes of the invention:
4-(2-{2-(Difluoromethoxy)-5-[5-methyl-5-(1,3,4-oxadiazol-2-yl)-4,5-dihydroisoxazol-3-yl]phenoxy}ethyl)morpholine (compound no. 13),
2-{2-methoxy-5-[5-methyl-5-(1,3,4-oxadiazol-2-yl)-4,5-dihydroisoxazol-3-yl]phenoxy}acetamide (compound no. 14),
   pharmaceutically acceptable salts, pharmaceutically acceptable solvates, enantiomers, diastereomers or N-oxides thereof.

Where desired, the compounds of Formula I and/or their pharmaceutically acceptable salts, pharmaceutically acceptable solvates, enantiomers, diastereomers or N-oxides may be advantageously used in combination with one or more other therapeutic agents. Examples of other therapeutic agents, which may be used in combination with compounds of Formula I of this invention and/or their pharmaceutically acceptable salts, pharmaceutically acceptable solvates, enantiomers, diastereomers or N-oxides include one other active ingredients selected from corticosteroids, β2-agonist, leukotriene antagonists, 5-lipoxygenase inhibitors, chemokine inhibitors, muscarinic receptor antagonists, p38 MAP kinase inhibitors, anticholinergics, antiallergics, PAP antagonists, EGFR kinase inhibitors, additional PDE-IV inhibitors, kinase inhibitors or combinations thereof.

The one or more β2-agonist as described herein may be chosen from those described in the art. The β2-agonists my include one or more compounds described in U.S. Pat. Nos. 3,705,233; 3,644,353; 3,642,896; 3,700,681; 4,579,985; 3,994,974; 3,937,838; 4,419,364; 5,126,375; 5,243,076; 4,992,474; and 4,011,258.

β2-agonists include, for example, one or more of albuterol, salbutamol, biltolterol, pirbuterol, levosalbutamol, tulobuterol, terbutaline, bambuterol, metaproterenol, fenoterol, salmeterol, carmoterol, arformoterol, formoterol, and their pharmaceutically acceptable salts or solvates thereof.

Corticosteroids as described herein may be chosen from those described in the art. Corticosteroids may be include one or more compounds described in U.S. Pat. Nos. 3,312,590; 3,983,233; 3,929,768; 3,721,687; 3,436,389; 3,506,694; 3,639,434; 3,992,534; 3,928,326; 3,980,778; 3,780,177; 3,652,554; 3,947,478; 4,076,708; 4,124,707; 4,158,055; 4,298,604; 4,335,121; 4,081,541; 4,226,862; 4,290,962; 4,587,236; 4,472,392; 4,472,393; 4,242,334; 4,014,909; 4,098,803; 4,619,921; 5,482,934; 5,837,699; 5,889,015; 5,278,156; 5,015,746; 5,976,573; 6,337,324; 6,057,307; 6,723,713; 6,127,353; and 6,180,781. The disclosures of these patents are incorporated herein by reference in their entireties.

Corticosteroids may include, for example, one or more of alclometasone, amcinonide, amelometasone, beclometasone, betamethasone, budesonide, ciclesonide, clobetasol, cloticasone, cyclomethasone, deflazacort, deprodone, dexbudesonide, diflorasone, difluprednate, fluticasone, flunisolide, halometasone, halopredone, hydrocortisone, hydrocortisone, methylprednisolone, mometasone, prednicarbate, prednisolone, rimexolone, tixocortol, triamcinolone, tolterodine, oxybutynin, ulobetasol, rofleponide, GW 215864, KSR 592, ST-126, dexamethasone and pharmaceutically acceptable salts, solvates thereof. Preferred corticosteroids include, for example, flunisolide, beclomethasone, triamcinolone, budesonide, fluticasone, mometasone, ciclesonide, and dexamethasone, while budesonide, fluticasone, mometasone, ciclesonide. Examples of possible salts or derivatives include: sodium salts, sulfobenzoates, phosphates, isonicotinates, acetates, propionates, dihydrogen phosphates, palmitates, pivalates, or furoates. In some cases, the corticosteroids may also occur in the form of their hydrates.

Muscarinic receptor antagonists include substances that directly or indirectly block activation of muscarinic cholinergic receptors. Examples include, but are not limited to, quaternary amines (e.g., methantheline, ipratropium, propantheline), tertiary amines (e.g., dicyclomine, scopolamine) and tricyclic amines (e.g., telenzepine). Other suitable muscarinic receptor antagonists include benztropine (commercially available as COGENTIN from Merck), hexahydro-sila-difenidol hydrochloride (HHSID hydrochloride disclosed in Lambrecht et al., Trends in Pharmacol. Sci., 10(Suppl):60 (1989); (±)-3-quinuclidinyl xanthene-9-carboxylate hemioxalate (QNX-hemioxalate; Birdsall et al., Trends in Pharmacol. Sci., 4:459 (1983); telenzepine dihydrochloride (Coruzzi et al., Arch. Int. Pharmacodyn. Ther., 302:232 (1989); and Kawashima et al., Gen. Pharmacol., 21:17 (1990)), and atropine.

Anticholinergics include, for example, tiotropium salts, ipratropium salts, oxitropium salts, salts of the compounds known from WO 02/32899: tropenol N-methyl-2,2-diphenylpropionate, scopine N-methyl-2,2-diphenylpropionate, scopine N-methyl-2-fluoro-2,2-diphenylacetate and tropenol N-methyl-2-fluoro-2,2-diphenylacetate; as well as salts of the compounds known from WO 02/32898: tropenol N-methyl-3,3′,4,4′-tetrafluorobenzilate, scopine N-methyl-3,3′,4,4′-tetrafluorobenzilate, scopine N-methyl-4,4′-dichlorobenzilate, scopine N-methyl-4,4′-difluorobenzilate, tropenol N-methyl-3,3′-difluorobenzilate, scopine N-methyl-3,3′-difluorobenzilate, and tropenol N-ethyl-4,4′-difluorobenzilate, optionally in the form of their hydrates and solvates. By salts are meant those compounds which contain, in addition to the above mentioned cations, as counter-ion, an anion with a single negative charge selected from among the chloride, bromide, and methanesulfonate.

Antiallergic agents include, for example, epinastine, cetirizine, azelastine, fexofenadine, levocabastine, loratadine, mizolastine, ketotifene, emedastine, dimetindene, clemastine, bamipine, hexachloropheniramine, pheniramine, doxylamine, chlorophenoxamine, dimenhydrinate, diphenhydramine, promethazine, ebastine, desloratadine, and meclizine. Preferred antiallergic agents include, for example, epinastine, cetirizine, azelastine, fexofenadine, levocabastine, loratadine, ebastine, desloratadine, and mizolastine, epinastine. Any reference to the above-mentioned antiallergic agents also includes any pharmacologically acceptable acid addition salts thereof, which may exist.

PAF antagonists include, for example, 4-(2-chlorophenyl)-9-methyl-2-[3-(4-morpholinyl)-3-propanon-1-yl]-6H-thieno[3,2-f][1,2,4]triazolo[4,3-α][1,4]diazepine and 6-(2-chlorophenyl)-8,9-dihydro-1-methyl-8-[(4-morpholinyl)carbonyl]-4H,7H-cyclopenta[4.5]thieno[3,2-f][1,2,4]triazolo[4,3-a][1,4]diazepine.

EGFR kinase inhibitors include, for example, 4-[(3-chloro-4-fluorophenyl)amino]-7-(2-{4-[(S)-(2-oxotetrahydrofuran-5-yl)carbonyl]piperazin-1-yl}-ethoxy)-6-[(vinylcarbonyl)amino]quinazoline, 4-[(3-chloro4-fluorophenyl)amino]-7-[4-((S)-6-methyl-2-oxomorpholin-4-yl)butyloxy]-6-[(vinylcarbonyl)amino]quinazoline, 4-[(3-chloro4-fluorophenyl)amino]-7-[4-((R-6-methyl-2-oxomorpholin-4-yl)butyloxy]-6-[(vinylcarbonyl)amino]quinazoline, 4-[(3-chloro-4-fluorophenyl)amino]-7-[2-((S)-6-methyl-2-oxomorpholin-4-yl)ethoxy]-6-[(vinylcarbonyl)amino]quinazoline, 4-[(3-chloro-4-fluorophenyl)amino]-6-[(4-{N-[2-(ethoxycarbonyl)ethyl]-N-[(ethoxycarbonyl)methyl]-amino}-1-oxo-2-buten-1-yl)amino]-7-cyclopropylmethoxyquinazoline, 4-[(R)-(1-phenylethyl)amino]-6-{[4-(morpholin-4-yl)-1-oxo-2-buten-1-yl]amino}-7-cyclopropyl-methoxyquinazoline, and 4-[(3-chloro-4-fluorophenyl)amino]-6-[3-(morpholin-4-yl)propyloxy]-7-methoxyquinazoline. Any reference to the above-mentioned EGFR kinase inhibitors also includes any pharmacologically acceptable acid addition salts thereof which may exist. By the physiologically or pharmacologically acceptable acid addition salts thereof which may be formed by the EGFR kinase inhibitors are meant, according to the invention, pharmaceutically acceptable salts selected from among the salts of hydrochloric acid, hydrobromic acid, sulfuric acid, phosphoric acid, methanesulfonic acid, acetic acid, fumaric acid, succinic acid, lactic acid, citric acid, tartaric acid, or maleic acid. The salts of the EGFR kinase inhibitors selected from among the salts of acetic acid, hydrochloric acid, hydrobromic acid, sulfuric acid, phosphoric acid, and methanesulfonic acid are preferred according to the invention.

p38 kinase inhibitors include, for example, 1-[5-tert-butyl-2-p-tolyl-2H-pyrazol-3-yl]-3-[4-(2-morpholin-4-ylethoxy)naphthalen-1-yl]urea; 1-[5-tert-butyl-2-p-tolyl-2H-pyrazol-3-yl]-3-[4-(2-(1-oxothiomorpholin-4-yl)ethoxy)naphthalen-1-yl]urea; 1-[5-tert-butyl-2-(2-methylpyridin-5-yl)-2H-pyrazol-3-yl]-3-[4-(2-pyridin-4-ylethoxy)naphtalen-1-yl]urea; 1-[5-tert-butyl-2-(2-methoxypyridin-5-yl)-2H-pyrazol-3-yl]-3-[4-(2-morpholin-4-ylethoxy)naphthalen-1-yl]urea; and 1-[5-tert-butyl-2-methyl-2H-pyrazol-3-yl]-3-[4-(2-morpholin-4-ylethoxy)naphthalen-1-yl]urea disclosed in our co-pending U.S. patent application No. 60/605,344; 4-[7-Oxo-8-(tetrahydro-pyran-4-yl)-6-o-tolyl-7,8-dihydro-pyrido[2,3-d]pyrimidin-2-ylamino]-piperidine-1-carboxylic acid tert-butyl ester; Hydrochloride salt of 2-(Piperidin-4-ylamino)-8-(tetrahydro-pyran-4-yl)-6-o-tolyl-8H-pyrido[2,3-d]pyrimidin-7-one; 2-(1-Methanesulfonyl-piperidin-4-ylamino)-8-(tetrahydro-pyran-4-yl)-6-o-totyl-8H-pyrido[2,3-d]pyrimidin-7-one; 2-(1-Benzyl-piperidin-4-ylamino)-8-(tetrahydro-pyran-4-yl)-6-o-tolyl-8H-pyrido[2,3-d]pyrimidin-7-one; 2-(1-Methyl-piperidin-4-ylamino)-8-(tetrahydro-pyran-4-yl)-6-o-tolyl-8H-pyrido[2,3-d]pyrimidin-7-one; 2-(4-Methyl-piperazin-1-ylamino)-8-(tetrahydro-pyran-4-yl)-6-o-tolyl-8H-pyrido[2,3-d]pyrimidin-7-one; 4-[6-(2-Chloro-phenyl)-7-oxo-8-(tetrahydro-pyran-4-yl)-7,8-dihydro-pyrido[2,3-d]pyrimidin-2-ylamino]-piperidine-1-carboxylic acid tert-butyl ester; 2-(Piperidin-1-ylamino)-8-(tetrahydro-pyran-4-yl)-6-o-tolyl-8H-pyrido[2,3-d]pyrimidin-7-one; 2-Cyclobutylamino-8-(tetrahydro-pyran-4-yl)-6-o-tolyl-8H-pyrido[2,3-d]pyrimidin-7-one; 2-(1-Acetyl-piperidin-4-ylamino)-8-(tetrahydro-pyran-4-yl)-6-o-tolyl-8H-pyrido[2,3-d]pyrimidin-7-one; 2-(1-Benzoyl-piperidin-4-ylamino)-8-(tetrahydro-pyran-4-yl)-6-o-tolyl-8H-pyrido[2,3-d]pyrimidin-7-one; 2-(1-Benzoyl-piperidin-4-ylamino)-8-(tetrahydro-pyran-4-yl)-6-o-tolyl-8H-pyrido[2,3-d]pyrimidin-7-one; 4-[7-Oxo-8-(tetrahydro-pyran-4-yl)-6-o-tolyl-7,8-dihydro-pyrido[2,3-d]pyrimidin-2-ylamino]-piperidine-1-carboxylic acid (4-fluoro-phenyl)-amide; 2-(1-Ethanesulfonyl-piperidin-4-ylamino)-8-(tetrahydro-pyran-4-yl)-6-o-tolyl-8H-pyrido[2,3-d]pyrimidin-7-one; 4-[7-Oxo-8-(tetrahydro-pyran-4-yl)-6-o-tolyl-7,8-dihydro-pyrido[2,3-d]pyrimidin-2-ylamino]-piperidine-1-carbothioic acid (4-fluoro-phenyl)-amide; 4-[7-Oxo-8-(tetrahydro-pyran-4-yl)-6-o-tolyl-7,8-dihydro-pyrido[2,3-d]pyrimidin-2-ylamino]-piperidine-1-carboxylic acid (4-trifluoromethyl-phenyl)-amide; 2-[4-(Propane-2-sulfonyl)-piperazin-1-ylamino]-8-(tetrahydro-pyran-4-yl)-6-o-tolyl-8H-pyrido[2,3-d]pyrimidin-7-one; 4-[7-Oxo-8-(tetrahydro-pyran-4-yl)-6-o-tolyl-7,8-dihydro-pyrido[2,3-d]pyrimidin-2-ylamino]-piperazine-1-carboxylic acid propylamide; 4-[7-Oxo-8-(tetrahydro-pyran-4-yl)-6-o-tolyl-7,8-dihydro-pyrido[2,3-d]pyrimidin-2-ylamino]-piperazine-1-carboxylic acid ((R)-1,2-dimethyl-propyl)-amide; 4-[7-Oxo-8-(tetrahydro-pyran-4-yl)-6-o-tolyl-7,8-dihydro-pyrido[2,3-d]pyrimidin-2-ylamino]-piperazine-1-carboxylic acid cyclohexylamide; 4-[7-Oxo-8-(tetrahydro-pyran-4-yl)-6-o-tolyl-7,8-dihydro-pyrido[2,3-d]pyrimidin-2-ylamino]-piperazine-1-carboxylic acid (4-fluoro-phenyl)-amide; 4-[7-Oxo-8-(tetrahydro-pyran-4-yl)-6-o-tolyl-7,8-dihydro-pyrido[2,3-d]pyrimidin-2-ylamino]-piperazine-1-carboxylic acid cyclopentyl methyl-amide; and the compounds which are disclosed in our co-pending U.S. patent application No. 60/598621, 60/630,517 and Indian patent application no 1098/DEL/2005 and 211/DEL/2005. Any reference to the above mentioned p38 kinase inhibitors also includes any pharmacologically acceptable acid addition salts thereof which may exist. By the physiologically or pharmacologically acceptable acid addition salts thereof which may be formed by the p38 kinase inhibitors are meant, according to the invention, pharmaceutically acceptable salts selected from among the salts of hydrochloric acid, hydrobromic acid, sulfuric acid, phosphoric acid, methanesulfonic acid, acetic acid, fumaric acid, succinic acid, lactic acid, citric acid, tartaric acid, and maleic acid.

Additional PDE-IV inhibitors include, for example, enprofylline, roflumilast, oglemilast, ariflo, Bay-198004, CP-325,366, BY343, D-4396 (Sch-351591), V-11294A, Z-15370, and AWD-12-281. Preferred PDE-IV inhibitors are selected from among enprofylline, roflumilast, ariflo, Z15370, and AWD-12-281. Any reference to the above mentioned PDE-IV inhibitors also includes any pharmacologically acceptable acid addition salts thereof which may exist. By the physiologically acceptable acid addition salts which may be formed by the above mentioned PDE-IV inhibitors are meant, according to the invention, pharmaceutically acceptable salts selected from among the salts of hydrochloric acid, hydrobromic acid, sulfuric acid, phosphoric acid, methanesulfonic acid, acetic acid, fumaric acid, succinic acid, lactic acid, citric acid, tartaric acid, or maleic acid. According to the invention, the salts selected from among the acetate, hydrochloride, hydrobromide, sulfate, phosphate, and methanesulfonate are preferred in this context.

The leukotriene antagonist can be selected from compounds not limited to those described in U.S. Pat. No. 5,565,473, U.S. Pat. No. 5,583,152, U.S. Pat. No. 4,859,692 or U.S. Pat. No. 4,780,469.

Examples of leukotriene antagonist include, but are not limited to, montelukast, zafirlukast, pranlukast and pharmaceutically acceptable salts thereof.

5-Lipoxygenase inhibitors can be selected from the compounds disclosed in U.S. Pat. Nos. 4,826,868, 4,873,259, EP 419049, EP 542356 or EP 542355. Examples may include but are not limited to atreleuton, zyflo (zileuton), ABT-761, fenleuton or tepoxalin.

Chemokine inhibitors can be selected from the compounds disclosed in EP 287436, EP 389359, EP 988292, WO 02/26723 or WO 01/90106.

Examples of chemokine inhibitors include, but are not limited to AMD3100, AZD 8309, BX-471, GW-766994, UK-427857, CP-481715, UK-107543, UK-382055 or UK-395859.

Examples set forth below demonstrate the synthetic procedures for the preparation of the representative compounds. The examples are provided to illustrate particular aspect of the disclosure and do not constrain the scope of the present invention as defined by the claims.

### Experimental Details

### Example 1

### Preparation of 3-hydroxy-4-difluoromethoxybenzaldehyde

Benzyltriethyl ammonium chloride (4.12 g, 0.0182 mol) was added to a solution of 3,4-dihydroxy benzaldehyde (5 g, 0.0362 mol) in dimethylformamide (35 mL). Sodium hydroxide solution (0.0905 mol of 30% solution) was added dropwise to the resulting reaction mixture for about 10 minutes with a continuous flow of chloro-difluoro methane. The reaction mixture was acidified with dilute hydrochloric acid and then diluted with water. It was extracted with ethyl acetate, washed with saturated solution of sodium chloride and concentrated under reduced pressure. The residue thus obtained was purified by column chromatography using 10% ethyl acetate in hexane to furmish the title compound. Yield: 2.5 g (37%).

### Example 2

### Preparation of 3-benzyloxy-4-methoxybenzaldehyde

The title compound was prepared according to the method described in J. Med. Chem. 1994, 37, 1696-1703.

The following compound was prepared by following above procedure:

3-(Benzyloxy)-4-(difluoromethoxy)benzaldehyde

Yield: 99%

### Example 3

### Preparation of 3-benzyloxy-4-methoxybenzaldehyde oxime

Hydroxylamine hydrochloride (50.25 g, 0.723 mol) and sodium acetate (59.31 g, 0.723 mmol) were added to a stirred solution of 3-benzyloxy-4-methoxybenzaldehyde (35 g, 0.144 mol) (example 2) in ethanol (200 mL). The reaction mixture was allowed to stir at room temperature for about 50 minutes. Ethanol was removed under reduced pressure, residue was poured in water (250 mL) and extraction was done with ethyl acetate (2×150 mL). Ethyl acetate layer was dried over anhydrous sodium sulphate, filtered and finally concentrated under reduced pressure to afford title compound. Yield: 36 g (96.8%).

The following compound was prepared by following above procedure:

3-(Benzyloxy)-4-(difluoromethoxy)benzaldehyde oxime

Yield: 99%

### Example 4

### Preparation of methyl 3-[3-(benzyloxy)-4-methoxyphenyl]-5-methyl-4,5-dihydroisoxazole-5-carboxylate

3-Benzyloxy-4-methoxybenzaldehyde oxime (10 g, 0.0389 mol) (example 3) was taken in tetrahydrofuran (50 mL). Methyl methacrylate (8.3 mL, 0.0778 mol) was added at room temperature. Sodium hypochlorite solution (100 mL) was added dropwise. The reaction mixture was stirred vigorously for about 14 hours at an ambient temperature. Tetrahydrofuran was removed under reduced pressure. Water was added and extraction was done with ethyl acetate. The organic layer was dried and concentrated in vacuo. The residue was purified by column chromatography using ethyl acetate and hexane (30:70).

Yield: 12.5 g (93.6%)

The following compounds were prepared by following above procedure:

Methyl 3-[3-(benzyloxy)-4-(difluoromethoxy)phenyl]-5-methyl-4,5-dihydroisoxazole-5-carboxylate

Yield: 81%

Methyl 3-[4-(difluoromethoxy)-3-ethoxyphenyl]-5-methyl-4,5-dihydroisoxazole-5-carboxylate

Yield: 83.2%

### Example 5

### Preparation of 3-[3-(benzyloxy)-4-methoxyphenyl]-5-methyl-4-5-dihydroisoxazole-5-carbohydrazide

Hydrazine-hydrate (10 mL) was added to methyl 3-[3-(benzyloxy)-4-methoxyphenyl]-5-methyl-4,5-dihydroisoxazole-5-carboxylate (1.0 g, 0.0029 mol) (example 4). The reaction mixture was heated overnight at about 120° C. It was cooled, water was added and extraction was done with ethyl acetate. The organic layer was dried and concentrated in vacuo.

Yield: 800 mg (77%)

The following compound was prepared by following above procedure:

3-[3-(Benzyloxy)-4-(difluoromethoxy)phenyl]-5-methyl-4,5-dihydroisoxazole-5-carbohydrazide

Yield: 89%

### Example 6

### Preparation of 2-{3-[3-(benzyloxy)-4-methoxyphenyl]-5-methyl-4,5-dihydroisoxazol-5-yl]-1,3,4-oxadiazole (compound no. 8)

Triethylorthoformate (5 mL) was added to 3-[3-(benzyloxy)-4-methoxyphenyl]-5-methyl-4,5-dihydroisoxazole-5-carbohydrazide (200 mg) (example 5). The reaction mixture was heated at about 120° C. for about 3 hours. Excess triethylorthoformate was evaporated and the residue was heated at about 140° C. for about 2 hours. The reaction mixture was diluted with water, saturated with potassium carbonate and extracted with ethyl acetate. The organic layer was dried, concentrated and purified by column chromatography (ethyl acetate:hexane:: 70:30).

Yield: 150 mg (73%).

¹HNMR: (CDCl₃): 2.04 (s, 3H), 3.39-3.44 (d, 1H), 3.92 (s, 3H), 4.16-4.21 (d, 1H), 5.16 (s, 3H), 6.88 (d, 1H, ArH), 7.09-7.12 (m, 1H, ArH), 7.26-7.46 (m, 6H, ArH), 8.43 (s, 1H).

The following compound was prepared by following the above procedure:

2-{3-[3-(Benzyloxy)-4-(difluoromethoxy)phenyl]-5-methyl-4,5-dihydroisoxazol-5-yl}-1,3,4-oxadiazole (compound no. 1)

Yield: 57%

### Example 7

### Preparation of 2-methoxy-5-[5-methyl-5-(1,3,4-oxadiazol-2-yl)-4,5-dihydroisoxazol-3-yl]phenol (compound no. 4)

Palladium/carbon (200 mg) was added to a solution of 2-[3-(3-benzyloxy-4-methoxyphenyl)-5-methyl-4,5-dihydroisoxazol-5-yl]-[1,3,4]oxadiazole (150 mg) (example 6) in methanol (5 mL). Hydrogen gas was perged through balloon. The reaction mixture was stirred in hydrogen atmosphere for about 3-4 hrs at room temperature. The catalyst palladium/carbon was filtered through celite and the mixture was washed with methanol. The organic solvent was concentrated under vacuo to give title compound.

Yield: 30 mg (29%).

¹HNMR: (CDCl₃): 1.97 (s, 3H), 3.42-3.46 (d, 1H), 3.93 (s, 3H), 4.18-4.22 (d, 1H), 6.87 (d, 1H), 7.17-7.20 (m, 1H), 7.29 (s, 1H), 8.44 (s, 1H).

M⁺+1: 276.2

The following compound was prepared by following above procedure:

2-Difluoromethoxy)-5-[5-methyl-5-(1,3,4-oxadiazol-2-yl)-4,5-dihydroisoxazol-3-yl]phenol (compound no. 2)

Yield: 20%

¹HNMR: (MeOD): 1.931 (s, 3H), 3.59-3.63 (d, 1H), 4.10-4.15 (d, 1H), 6.65-7.02 (t, 1H), 7.13-7.18 (m, 2H), 7.3 (s, 1H), 8.99 (s, 1H).

M⁺+1: 312.1

### Example 8

### Preparation of ethyl {2-methoxy-5-[5-methyl-5-(1,3,4-oxadiazol-2-yl)-4,5-dihydroisoxazol-3-yl]phenoxy}acetate (compound no. 3)

Potassium carbonate (100 mg, 0.00072 mol) and bromoethyl acetate (0.05 mL, 0.00043 mol) were added to 2-methoxy-5-[5-methyl-5-(1,3,4-oxadiazol-2-yl)-4,5-dihydroisoxazol-3-yl]phenol (80 mg, 0.00029 mol) (example 7) in dimethylformamide (1 mL). The reaction mixture was stirred overnight at room temperature. Water was added and the extraction was done with ethyl acetate. The organic layer was dried over anhydrous sodium sulphate, concentrated in vacuo and the residue was purified by column chromatography (ethyl acetate:hexane::50:50).

Yield: 50 mg (48%).

¹HNMR: (CDCl₃): 1.24-1.31 (t, 3H), 1.97 (s, 3H), 3.42-3.46 (d, 1H), 4.09 (s, 3H), 4.11-4.15 (d, 1H), 4.22-4.29 (q, 2H), 4.71 (s, 2H), 6.91 (d, 1H), 7.15 (d, H), 7.30 (s, 1H), 8.44 (s, 1H).

M⁺+1: 362.1

The following compounds were prepared by following above procedure:

Ethyl {2-(difluoromethoxy)-5-[5-methyl-5-(1,3,4-oxadiazol-2-yl)-4,5-dihydroisoxazol-3-yl]phenoxy}acetate (compound no. 5)

¹HNMR: (CDCl₃): 1.26-1.32 (t, 3H), 1.98 (s, 3H), 3.45 (d, 1H), 4.20-4.29 (m, 3H), 4.75 (s, 1H), 6.58-6.96 (t, 1H), 7.14 (d, 1H), 7.23-7.27 (m, 1H), 7.38 (s, 1H), 8.46 (s, 1H).

M⁺+1: 397.87

2-{2-Methoxy-5-[5-methyl-5-(1,3,4-oxadiazol-2-yl)-4,5-dihydroisoxazol-3-yl]phenoxy}ethanol (compound no. 6)

¹HNMR: (CD₃OD): 1.91 (s, 3H), 3.58 (d, 1H), 3.87 (d, 1H), 3.91 (s, 3H), 4.07-4.17 (m, 4H), 6.97 (d, 1H), 7.24 (d, 1H), 7.37 (s, 1H), 8.97 (s, 1H).

M⁺+1: 320.0

2-{2-Difluoromethoxy)-5-[5-methyl-5-(1,3,4-oxadiazol-2-yl)-4,5-dihydroisoxazol-3-yl]phenoxy}ethanol (compound no. 10)

¹HNMR: (CDCl₃): 1.98 (s, 3H), 2.91 (d, 1H), 3.45 (d, 1H), 3.98-4.01 (t, 2H), 4.16-4.19 (t, 2H), 6.36-6.85 (t, 1H), 7.11-7.14 (m, 1H), 7.20-7.26 (m, 1H), 7.47 (s, 1H), 8.45 (s, 1H).

M⁺+1: 355.98

4-(2-{2-Methoxy-5-[5-methyl-5-(1,3,4-oxadiazol-2-yl)-4,5-dihydroisoxazol-3-yl]phenoxy}ethyl)morpholine (compound no. 7)

¹HNMR: (CDCl₃): 1.98 (s, 3H), 2.64 (bs, 4H), 2.89-2.91 (m, 2H), 3.44-3.48 (d, 1H), 3.74-3.77 (m, 4H), 3.90 (s, 3H), 4.19-4.25 (m, 3H), 6.87-6.89 (d, 1H), 7.10 (d, 1H), 7.39 (s, 1H), 8.45 (s, 1H).

M⁺+1: 389.05

The following compound can be prepared by following above procedure:

4-(2-{2-(Difluoromethoxy)-5-[5-methyl-5-(1,3,4-oxadiazol-2-yl)-4,5-dihydroisoxazol-3-yl]phenoxy}ethyl)morpholine (compound no. 13).

### Example 9

### Preparation of 2-{2-(difluoromethoxy)-5-[5-methyl-5-(1,3,4-oxadiazol-2-yl)-4,5-dihydroisoxazol-3-yl]phenoxy}acetamide (compound no. 9)

Methanolic ammonia (5 mL) was added to ethyl {2-(difluoromethoxy)-5-[5-methyl-5-(1,3,4-oxadiazol-2-yl)-4,5-dihydroisoxazol-3-yl]phenoxy}acetate (30 mg) (example 8). The reaction mixture was stirred overnight at room temperature. Methanol was evaporated off and the residue was purified by preparative thin layer chromatography using ethyl acetate.

Yield 15 mg (54%).

¹HNMR: (CDCl₃): 1.99 (s, 3H), 3.45 (d, 1H), 4.25 (d, 1H), 4.57 (s, 2H), 5.86 (bs, 1H), 6.31-6.80 (t, 1H), 7.21-7.26 (m, 3H), 8.45 (s, 1H).

M⁺+1: 368.98

The following compound can be prepared by following above method:

2-{2-methoxy-5-[5-methyl-5-(1,3,4-oxadiazol-2-yl)-4,5-dihydroisoxazol-3-yl]phenoxy}acetamide (compound no. 14).

### Example 10

### Preparation of 3-[4-(difluoromethoxy)-3-ethoxyphenyl]-5-methyl-4,5-dihydroisoxazole-5-carboxylic acid

Methyl 3-[4-(difluoromethoxy)-3-ethoxyphenyl]-5-methyl-4,5-dihydroisoxazole-5-carboxylate (1 g, 0.0030 mol) (example 4) was taken in tetrahydrofuran (10 mL). Lithium hydroxide solution (382 mg, 0.0091 mol in 1 mL water) was added and the reaction mixture was stirred at room temperature overnight. Tetrahydrofuran was removed under reduced pressure. Water was added and the mixture was extracted with ethyl acetate. Aqueous layer was acidified by adding concentrated hydrochloric acid and extracted with ethyl acetate. The organic layer was dried over anhydrous sodium sulphate and concentrated in vacuo to give title compound.

Yield: 1 g (crude)

### Example 11

### Preparation of (5R or 5S)-3-[4-(difluoromethoxy)-3-ethoxyphenyl]-5-methyl-N-[(1R)-1-phenylethyl]-4,5-dihydroisoxazole-5-carboxamide

3-[4-(Difluoromethoxy)-3-ethoxyphenyl]-5-methyl-4,5-dihydroisoxazole-5-carboxylic acid (500 mg, 0.00158 mol) (example 10) and oxalyl chloride (0.413 mL, 0.00474 mol) in benzene (15 mL) were refluxed for about half an hour. Benzene was evaporated off and the residue was diluted with dichloromethane (10 mL). The solution obtained was added to the solution of (s) (−) alpha methyl benzyl amine (0.425 mL, 0.00316 mol) in dichloromethane (10 mL) dropwise at about 0° C. The reaction mixture was stirred for about 1 hr at room temperature. The organic layer was washed with 1N hydrochloric acid and then with 10% sodium bicarbonate. It was extracted with water, dried and concentrated in vacuo to give mixture of title diastereomers (I and II). Diastereomers were then separated by column chromatography.

Yield (total): 67.8%

¹NMR: diastereomer I: (CDCl₃): 1.44 (m, 6H), 1.68 (s, 3H), 3.20 (d, 1H), 4.00 (d, 1H), 4.12-4.17 (q, 2H), 5.03-5.08 (m, 1H), 6.37-6.87 (t, 1H), 7.06-7.38 (m, 8H).

Mass: 419.02 (M⁺+1)

¹HNMR: diastereomer II: (CDCl₃): 1.42-1.52 (m, 6H), 1.74 (s, 3H), 3.19 (d, 1H), 3.74 (d, 1H), 4.07-4.14 (q, 2H), 5.05 (m, 1H), 6.35-6.85 (t, 1H), 7.01-7.32 (m, 8H).

Mass: 419.02 (M⁺+1)

### Example 12

### Preparation of (5R or 5S)-3-(3-ethoxy-4-hydroxyphenyl)-5-methyl-4,5-dihydroisoxazole-5-carboxylic acid

(5R or 5S)-3-[4-(difluoromethoxy)-3-ethoxyphenyl]-5-methyl-N-[(1R)-1-phenylethyl]-4,5-dihydroisoxazole-5-carboxamide (500 mg, 0.00119 mol) (example 11) was taken in a mixture of isopropanol:methanol (15:6 mL). Hydrazine hydrate (8.9 mL, 0.1794 mol) and potassium hydroxide (10 g, 0.1794 mol) were added to the mixture. The reaction mixture was heated at refluxing temperature for about 48 hrs. The organic solvent was removed under vacuo, water was added to the residue and it was acidified with concentrated hydrochloric acid. The extraction was done with ethyl acetate (2×50 mL) and the mixture was basified with saturated sodium bicarbonate solution and again extracted with ethyl acetate. The aqueous layer was acidified and extracted with ethyl acetate, dried and concentrated in vacuo to give respective enantiomers (enantiomer I and enantiomer II).

Enantiomer I: Yield: 200 mg (63%)

¹HNMR: (CD₃OD): 1.40-1.45 (t, 3H), 1.64 (s, 1H), 3.28-3.34 (m, 1H), 3.76 (d, 1H), 4.07-4.14 (q, 2H), 6.82 (d, 1H), 7.03-7.07 (m, 1H), 7.27 (s, 1H).

Mass: 266.08 (M⁺+1)

Enantiomer II:

¹HNMR: (MeOD+D₂O) 1.41-1.46 (t, 3H), 1.67 (s, 1H), 3.40 (d, 1H), 3.83 (d, 1H), 4.09-4.16 (q, 2H), 6.88 (d, 1H), 7.07-7.10 (m, 1H), 7.29 (s, 1H).

Mass: 266.05 (M⁺+1)

### Example 13

### Preparation of methyl (5R or 5S)-3-(3-ethoxy-4-hydroxyphenyl)-5-methyl-4,5-dihydroisoxazole-5-carboxylate

A mixture of (5R or 5S)-3-(3-ethoxy-4-hydroxyphenyl)-5-methyl-4,5-dihydroisoxazole-5-carboxylic acid (140 mg, 0.00052 mol) (example 12) and concentrated sulfuric acid (0.1 mL) in methanol was refluxed at 60-70° C. for about 3 hrs. Methanol was evaporated off. The reaction mixture was diluted with water, extracted with ethyl acetate and washed with 10% solution of sodium bicarbonate. The organic layer was dried over anhydrous sodium sulphate and concentrated in vacuo to give the respective enantiomers (enantiomer III and enantiomer IV).

Enanatiomer III: Yield: 115 mg (78%).

¹HNMR: (CDCl₃): 1.43-1.48 (t, 3H), 1.71 (s, 1H), 3.19 (d, 1H), 3.80 (s, 3H), 3.84 (d, 1H), 4.11-4.18 (q, 2H), 5.90 (bs, 1H), 6.89-6.97 (m, 2H), 7.37 (s, 1H).

Mass: 280.06 (M⁺+1)

Enanatiomer IV: Yield: 78%

¹HNMR: (CDCl₃): 1.44-1.47 (t, 3H), 1.71 (s, 1H), 3.19 (d, 1H), 3.80 (s, 3H), 3.84 (d, 1H), 4.12-4.17 (q, 2H), 5.90 (s, 1H), 6.90-6.99 (m, 2H), 7.36 (s, 1H).

Mass: 279.99 (M⁺+1)

### Example 14

### Preparation of methyl (5R or 5S)-3-[4-(difluoromethoxy)-3-ethoxyphenyl]-5-methyl-4,5-dihydroisoxazole-5-carboxylate

Freon gas was purged through a mixture of methyl (5R or 5S)-3-(3-ethoxy-4-hydroxyphenyl)-5-methyl-4,5-dihydroisoxazole-5-carboxylate (110 mg, 0.00039 mol) (example 13), potassium carbonate (245 mg, 0.00078 mol) and benzyltriethylammonium chloride (8.0 mg, 0.000039 mol) in dimethylformamide (5 mL) at about −10° C. for about 3-4 minutes. The reaction mixture was stirred overnight at room temperature. It was diluted with water and extracted with ethyl acetate. The organic layer was washed with brine, dried over anhydrous sodium sulphate and concentrated in vacuo to give respective enantiomers (enantiomer V and enantiomer VI).

Enantiomer V: Yield: 90 mg (70%).

¹HNMR: (CDCl₃): 1.43-1.48 (t, 3H), 1.72 (s, 1H), 3.19 (d, 1H), 3.81 (s, 1H), 3.86 (d, 1H), 4.09-4.16 (q, 2H), 6.36-6.86 (t, 1H), 7.00-7.03 (m, 1H), 7.17 (d, 1H), 7.43 (s, 1H).

Mass: 329.97 (M⁺+1)

Enantiomer VI: Yield: 70%

¹HNMR: (CDCl₃): 1.41-1.48 (t, 1H), 1.72 (s, 1H), 3.19 (d, 1H), 3.81 (s, 3H), 3.89 (d, 1H), 4.09-4.16 (q, 2H), 6.36-6.86 (t, 1H), 7.00-7.03 (m, 1H), 7.17 (d, 1H), 7.43 (s, 1H).

Mass: 329.97 (M⁺+1)

### Example 15

### Preparation of (5R or 5S)-3-[4-(difluoromethoxy)-3-ethoxyphenyl]-5-methyl-4,5-dihydroisoxazole-5-carbohydrazide

Hydrazine-hydrate (2 mL) was added to methyl (5R or 5S)-3-[4-(difluoromethoxy)-3-ethoxyphenyl]-5-methyl-4,5-dihydroisoxazole-5-carboxylate (80 mg, 0.000243 mol) (example 14). The reaction mixture was heated at about 120° C. for about 6 hrs. It was cooled and water was added. Extraction was done with ethyl acetate, the organic layer was dried and concentrated in vacuo to give respective enantiomers (enantiomer VII and enantiomer VIII).

Enantiomer VII: Yield: 60 mg (60%)

Enantiomer VIII: Yield: 75%

### Example 16

### Preparation of 2-{(5R or 5S)-3-[4-(difluoromethoxy)-3-ethoxyphenyl]-5-methyl-4,5-dihydroisoxazol-5-yl]-1,3,4-oxadiazole

Triethylorthoformate (2 mL) was added to (5R or 5S)-3-[4-(difluoromethoxy)-3-ethoxyphenyl]-5-methyl-4,5-dihydroisoxazole-5-carbohydrazide (60 mg, 0.000182 mol) (example 15). The reaction mixture was heated at about 120° C. for about 3 hours. Excess triethylorthoformate was evaporated and the residue was heated at about 140° C. for about 2 hours. The mixture was diluted with water, saturated with potassium carbonate and extracted with ethyl acetate. The organic layer was dried, concentrated and purified by column chromatography. The residue was purified on crystallization by using diisopropyl ether to give the respective enantiomers (enantiomer IX and enantiomer X).

Enantiomer IX: Yield: 15 mg (25%).

¹HNMR: (CDCl₃): 1.43-1.48 (t, 3H), 1.98 (s, 3H), 3.45 (d, 1H), 4.10-4.17 (q, 2H), 4.16 (d, 1H), 6.37-6.87 (t, 1H), 7.06-7.09 (m, 1H), 7.19 (d, 1H), 7.38 (s, 1H), 8.44 (s, 1H).

Chiral purity: 98.22%

Mass: 339.96 (M⁺+1)

Enantiomer X: Yield: 15%

¹HNMR: (CDCl₃): 1.44-1.48 (t, 3H), 1.98 (s, 3H), 3.45 (d, 1H), 4.07-4.14 (q, 2H), 4.19 (d, 1H), 6.37-6.87 (t, 1H), 7.06-7.09 (m, 1H), 7.20 (d, 1H), 7.44 (s, 1H), 8.44 (s, 1H).

Chiral purity: 98.85%

Mass: 339.96 (M⁺+1)

### Example 17

### Efficacy of Compounds

### (a) PDE-IV Enzyme Assay

The efficacy of compounds of PDE-IV inhibitors was determined by an enzyme assay using U937 cell cytosolic fraction (Biochem. Biophys. Res. Comm., 197: 1126-1131, 1993). The enzyme reaction was carried out in the presence of cAMP (1 μM) at 30° C. in the presence or absence of test compound for 45-60 min. An aliquot of this reaction mixture was taken further for the ELISA assay and the protocol of the kit followed to determine level of cAMP in the sample. The concentration of the cAMP in the sample directly correlated with the degree of PDE-4 enzyme inhibition. Results were expressed as percent control and the IC₅₀ values of test compounds were found to be in the range from about 10 μM to about 0.1 nM concentration.

### (b) Cell Based Assay for TNF-α Release

Method of Isolation of Human Peripheral Blood Mononuclear Cells (PBMNC):

Human whole blood was collected in vacutainer tubes containing heparin or EDTA as an anti coagulant. The blood was diluted (1:1) in sterile phosphate buffered saline and 10 mL was carefully layered over 5 mL Ficoll Hypaque gradient (density 1.077 g/mL) in a 15 mL conical centrifuge tube. The sample was centrifuged at 3000 rpm for 25 minutes in a swing-out rotor at room temperature. After centrifugation, interface of cells were collected, diluted at least 1:5 with PBS (phosphate buffered saline) and washed three times by centrifugation at 2500 rpm for 10 minutes at room temperature. The cells were resuspended in serum free RPMI 1640 medium at a concentration of 2 million cells/mL.

### LPS (Lipopolysaccharide) Stimulation of Human PBMNC:

PBMN cells (0.1 mL; 2 million/mL) were co-incubated with 20 μl of compound (final DMSO concentration of 0.2%) for 10 min in a flat bottom 96 well microtiter plate. Compounds were dissolved in DMSO initially and diluted in medium for a final concentration of 0.2% DMSO. LPS (1 μg/mL, final concentration) was then added at a volume of 10 μl per well. After 30 min, 20 μl of fetal calf serum (final concentration of 10%) was added to each well. Cultures were incubated overnight at 37° C. in an atmosphere of 5% CO₂ and 95% air. Supernatant were then removed and tested by ELISA for TNF-α release using a commercial kit (e.g. BD Biosciences). For whole blood, the plasma samples were diluted 1:20 for ELISA. The level of TNF-α in treated wells was compared with the vehicle treated controls and inhibitory potency of compound was expressed as IC₅₀ values calculated by using Graph pad prism. IC₅₀ values of test compounds were found to be in the range from about 10 μM to about 100 nM concentration.

$$Percent inhibition = 100 - \frac{Percent TNF \text{-} α drug treated}{Percent TNF \text{-} α in vehicle treated} \times 100$$

(c) In-Vitro Assay to Evaluate Efficacy of PDE IV Inhibitors in Combination with p38 MAP Kinase Inhibitors

Perform the assay as described in (b) above, with individual compounds and their combinations tested at sub-optimal doses.

(d) In-Vitro Assay to Evaluate Efficacy of PDE IV Inhibitors in Combination with β2-Agonists

Measurement of Intracellular cAMP Elevation in U937 Cells

Grow U937 cells (human promonocytic cell line) in endotoxin-free RPMI1640+HEPES medium containing 10% (v/v) heat-inactivated foetal bovine serum and 1% (v/v) of an antibiotic solution (5000 IU/mL penicillin, 5000 μg/mL streptomycin). Resuspend cells (0.25×10⁶/200 μl) in Krebs' buffer solution and incubate at 37° C. for 15 min in the presence of test compounds or vehicle (20 μl). Initiate generation of cAMP by adding 50 μl of 10 μM prostaglandin (PGE2). Stop the reaction after 15 min, by adding 1 N HCl (50 μl) and place on ice for 30 min. Centrifuge the sample (450 g, 3 min), and measure levels of cAMP in the supernatant using cAMP enzyme-linked immunosorbent assay kit (Assay Designs). Calculate percent inhibition by the following formula and calculate IC₅₀ value using Graph pad prism.

$$Percent inhibition = 100 - \frac{Percent conversion in drug treated}{Percent conversion in vehicle treated} \times 100$$

(e) In-Vitro Functional Assays to Evaluate Efficacy of PDE IV Inhibitors in Combination with Beta-Agonists

### Animals and Anaesthesia

Procure Guinea Pig (400-600 gm) and remove trachea under anesthesia (sodium pentobarbital, 300 mg/kg i.p) and immediately keep it in ice-cold Krebs Henseleit buffer. Indomethacin (10 μM) is present throughout the KH buffer to prevent the formation of bronchoactive prostanoids.

### Trachea Experiments:

Clean the tissue off adherent fascia and cut it into strips of equal size (with approx. 4-5 tracheal rings in each strip). Remove the epithelium by careful rubbing, minimizing damage to the smooth muscle. Open the trachea along the mid-dorsal surface with the smooth muscle band intact and make a series of transverse cuts from alternate sides so that they do not transect the preparation completely. Tie opposite end of the cut rings with the help of a thread. Mount the tissue in isolated tissue baths containing 10 mL Krebs Henseleit buffer maintained at 37° C. and bubbled with carbogen, at a basal tension of 1 g. Change the buffer 4-5 times for about an hour. Equilibrate the tissue for 1 hr with 1 μM carbachol or 10 μM histamine for stabilization. Wash it for 30 minutes followed by a precontraction with histamine (10 μM) or carbachol (1 μM). Allow the developed tension to stabilize for 15-20 minutes followed by the cumulative addition of beta-agonists prior to incubation with suboptimal dose of PDE IV inhibitor. Record the contractile response of tissues either on Powerlab data acquisition system or on Grass polygraph (Model 7). Express the relaxation as percentage of maximum carbachol response. Express the data as mean ±S.E. mean for n observations. Calculate the EC₅₀ as the concentration producing 50% of the maximum relaxation to 1 μM carbachol. Compare percent relaxation between the treated and control tissues using non-parametric unpaired t-test. A p value of <0.05 is considered to be statistically significant.

(i) In-Vivo Assay to Evaluate Efficacy of PDE IV Inhibitors in Combination with Beta-Agonists

Lipopolysaccharide (LPS) Induced Airway Hyperreactivity (AHR) and Neutrophilia:

Drug Treatment:

Beta-agonist (1 ng/kg to 1 mg/kg) and PDE4 inhibitor (1 ng/kg to 1 mg/kg) can be instilled intratracheally under anesthesia either alone or in combination.

Method:

Use male wistar rats weighing 200±20 gm in the study. Rats should have free access to food and water. On the day of experiment, expose animals to lipopolysaccharide (LPS, 100 μg/mL) for 40 min. Expose one group of vehicle treated rats to phosphate buffered saline (PBS) for 40 min. Two hours after LPS/PBS exposure, place animals inside a whole body plethysmograph (Buxco Electronics, USA) and expose to PBS or increasing acetylcholine (1, 6, 12, 24, 48 and 96 mg/mL) aerosol until Penh values (index of airway resistance) of rats attain 2 times the value (PC-100) seen with PBS alone. Record the respiratory parameters online using Biosystem XA software, (Buxco Electronics, USA). Express Penh, at any chosen dose of acetylcholine is, as percent of PBS response and using a nonlinear regression analysis compute PC100 (2 folds of PBS value) values. Calculate percent inhibition using the following formula.

$$\% Inhibition = \frac{PC {100}_{LPS} - PC {100}_{TEST}}{PC {100}_{LPS} - PC {100}_{PBS}} \times 100$$

### Where,

PC100_{LPS}=PC100 in vehicle treated group challenged group with LPS
PC100_{TEST}=PC100 in group treated with a given dose of test compound
PC100_{PBS}=PC100 in vehicle treated group challenged with PBS

Sacrifice animals immediately after recording the airway hyperreactivity response and perform bronchoalveolar lavage (BAL). Centrifuge the collected lavage fluid at 3000 rpm for 5 min, at 4° C. Collect pellet and resuspend in 1 mL HBSS. Perform total leukocyte count in the resuspended sample. Use a portion of suspension for cytocentrifugation and staining with Leishmann's stain for differential leukocyte count. Express total leukocyte and Neutrophil counts as cell count (millions cells mL⁻¹ of BAL). Compute percent inhibition using the following formula.

$$\% Inhibition = \frac{{NC}_{LPS} - {NC}_{TEST}}{{NC}_{LPS} - {NC}_{PBS}} \times 100$$

### Where,

NC_{LPS}=Percentage of neutrophil in vehicle treated group challenged with LPS
NC_{TEST}=Percentage of neutrophil in group treated with a given dose of test compound
NC_{PBS}=Percentage of neutrophil in vehicle treated group challenged with PBS

Compute ED₅₀ from percent inhibition values using Graph Pad Prism software (Graphpad Software Inc., USA).

(g) In-Vitro Functional Assay to Evaluate Efficacy of PDE-IV Inhibitors in Combination with Muscarinic Receptor Antagonists

Animals and Anaesthesia:

Procure Guinea Pig (400-600 gm) and remove trachea under anesthesia (sodium pentobarbital, 300 mg/kg i.p) and immediately keep in ice-cold Krebs Henseleit buffer. Indomethacin (10 uM) is present throughout the KH buffer to prevent the formation of bronchoactive prostanoids.

Trachea Experiments:

Clean the tissue off adherent fascia and cut it into strips of equal size (with approx. 4-5 tracheal rings in each strip). Remove the epithelium by careful rubbing, minimizing damage to the smooth muscle. Open the trachea along the mid-dorsal surface with the smooth muscle band intact and make a series of transverse cuts from alternate sides so that they do not transect the preparation completely. Tie opposite end of the cut rings with the help of a thread. Mount the tissue in isolated tissue baths containing 10 mL Krebs Henseleit buffer maintained at 37° C. and bubbled with carbogen, at a basal tension of 1 g. Change the buffer 4-5 times for about an hour. Equilibrate the tissue for 1 hr for stabilization. After 1 hr, challenge the tissue with 1 μM carbachol. Repeat this after every 2-3 washes till two similar consecutive responses are obtained. At the end of stabilization, wash the tissues for 30 minutes followed by incubation with suboptimal dose of MRA/Vehicle for 20 minutes prior to contraction of the tissues with 1 μM carbachol and subsequently assess the relaxant activity of the PDE-IV inhibitor [10⁻⁹ M to 10⁻⁴ M] on the stabilized developed tension/response. Record the contractile response of tissues either on Powerlab data acquisition system or on Grass polygraph (Model 7). Express the relaxation as percentage of maximum carbachol response. Express the data as mean±S.E. for n observations. Calculate the EC₅₀ as the concentration producing 50% of the maximum relaxation to 1 μM carbachol. Compare percent relaxation between the treated and control tissues using non-parametric unpaired t-test. A p value of <0.05 is considered to be statistically significant.

h) In-Vivo Assay to Evaluate Efficacy of PDE-IV inhibitors In Combination with MRA Inhibitors

Drug Treatment:

MRA (1 ng/kg to 1 mg/kg) and PDE-IV inhibitor (1 ng/kg to 1 mg/kg) can be instilled intratracheally under anesthesia either alone or in combination.

Method:

Use male wistar rats weighing 200±20 gm in the study. Rats should have free access to food and water. On the day of experiment, expose animals to lipopolysaccharide (LPS, 100 μg/mL) for 40 min. Expose one group of vehicle treated rats to phosphate buffered saline (PBS) for 40 min. Two hours after LPS/PBS exposure, place animals inside a whole body plethysmograph (Buxco Electronics, USA) and expose to PBS or increasing acetylcholine (1, 6, 12, 24, 48 and 96 mg/mL) aerosol until Penh values (index of airway resistance) of rats attain 2 times the value (PC-100) seen with PBS alone. Record the respiratory parameters online using Biosystem XA software, (Buxco Electronics, USA). Express Penh, at any chosen dose of acetylcholine is, as percent of PBS response and using a nonlinear regression analysis compute PC100 (2 folds of PBS value) values. Calculate percent inhibition using the following formula.

$$\% Inhibition = \frac{PC {100}_{LPS} - PC {100}_{TEST}}{PC {100}_{LPS} - PC {100}_{PBS}} \times 100$$

### Where,

PC100_{LPS}=PC100 in vehicle treated and LPS challenged group
PC100_{TEST}=PC100 in group treated with a given dose of test compound
PC100_{PBS}=PC100 in vehicle treated group challenged with PBS

Sacrifice animals immediately after recording the airway hyperreactivity response and perform bronchoalveolar lavage (BAL). Centrifuge the collected lavage fluid at 3000 rpm for 5 min, at 4° C. Collect pellet and resuspend in 1 mL HBSS. Perform total leukocyte count in the resuspended sample. Use a portion of suspension for cytocentrifugation and staining with Leishmann's stain for differential leukocyte count. Express total leukocyte and Neutrophil counts as cell count (millions cells mL⁻¹ of BAL). Compute percent inhibition using the following formula.

$$\% Inhibition = \frac{{NC}_{LPS} - {NC}_{TEST}}{{NC}_{LPS} - {NC}_{PBS}} \times 100$$

### Where,

NC_{LPS}=Percentage of neutrophil in vehicle treated group challenged with LPS
NC_{TEST}=Percentage of neutrophil in group treated with a given dose of test compound
NC_{PBS}=Percentage of neutrophil in vehicle treated group challenged with PBS

Compute ED₅₀ from percent inhibition values using Graph Pad Prism software (Graphpad Software Inc., USA).

We claim:

1. A compound having the structure of Formula I: 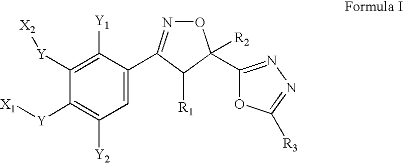
their pharmaceutically acceptable salts, pharmaceutically acceptable solvates, enantiomers, diastereomers or N-oxides, wherein
R₁, R₂ and R₃ are independently selected from hydrogen or alkyl;
X₁ and X₂ are independently selected from hydrogen, alkyl, alkenyl, alkynyl, cycloalkyl, aryl, aralkyl, heteroaryl, heterocyclyl, (heteroaryl)alkyl or (heterocyclyl)alkyl;
Y represents an oxygen atom, a sulphur atom, or NR (wherein R is selected from hydrogen, alkyl, alkenyl, alkynyl, un(saturated) cycloalkyl, aryl, aralkyl, heteroaryl, heterocyclyl, (heteroaryl)alkyl, or (heterocyclyl)alkyl);
Y₁ and Y₂ are independently selected from hydrogen, alkyl, nitro, cyano, halogen, OR (wherein R is the same as defined earlier), SR (wherein R is the same as defined earlier), NHR (wherein R is the same as defined earlier), COOR′ or COR′ (wherein R′ is hydrogen, alkyl, alkenyl, alkynyl, (un)saturated cycloalkyl, aryl, aralkyl, heterocyclyl, (heterocyclyl)alkyl, or (heteroaryl)alkyl);
Y₁ and X₂, X₁ and Y₂, X₁ and X₂ may together form a cyclic ring fused with the ring A containing 3-5 carbon atoms within the ring and having 1-3 heteroatoms selected from N, O or S.

2. A compound, which is selected from:
2-{3-[3-(Benzyloxy)-4-(difluoromethoxy)phenyl]-5-methyl-4,5-dihydroisoxazol-5-yl}-1,3,4-oxadiazole (compound no. 1),
2-(Difluoromethoxy)-5-[5-methyl-5-(1,3,4-oxadiazol-2-yl)-4,5-dihydroisoxazol-3-yl]phenol (compound no. 2),
Ethyl {2-methoxy-5-[5-methyl-5-(1,3,4-oxadiazol-2-yl)-4,5-dihydroisoxazol-3-yl]phenoxy}acetate (compound no. 3),
2-Methoxy-5-[5-methyl-5-(1,3,4-oxadiazol-2-yl)-4,5-dihydroisoxazol-3-yl]phenol (compound no. 4),
Ethyl {2-(difluoromethoxy)-5-[5-methyl-5-(1,3,4-oxadiazol-2-yl)-4,5-dihydroisoxazol-3-yl]phenoxy}acetate (compound no. 5),
2-{2-Methoxy-5-[5-methyl-5-(1,3,4-oxadiazol-2-yl)-4,5-dihydroisoxazol-3-yl]phenoxy}ethanol (compound no. 6),
4-(2-{2-Methoxy-5-[5-methyl-5-(1,3,4-oxadiazol-2-yl)-4,5-dihydroisoxazol-3-yl]phenoxy}ethyl)morpholine (compound no. 7),
2-{3-[3-(Benzyloxy)-4-methoxyphenyl]-5-methyl-4,5-dihydroisoxazol-5-yl}-1,3,4-oxadiazole (compound no. 8),
2-{2-(Difluoromethoxy)-5-[5-methyl-5-(1,3,4-oxadiazol-2-yl)-4,5-dihydroisoxazol-3-yl]phenoxy}acetamide (compound no. 9),
2-{2-(Difluoromethoxy)-5-[5-methyl-5-(1,3,4-oxadiazol-2-yl)-4,5-dihydroisoxazol-3-yl]phenoxy}ethanol (compound no. 10),
2-{(5S or 5R)-3-[4-(difluoromethoxy)-3-ethoxyphenyl]-5-methyl-4,5-dihydroisoxazol-5-yl}-1,3,4-oxadiazole (compound no. 11),
2-{(5R or 5S)-3-[4-(difluoromethoxy)-3-ethoxyphenyl]-5-methyl-4,5-dihydroisoxazol-5-yl}-1,3,4-oxadiazole (compound no. 12),
4-(2-{2-(Difluoromethoxy)-5-[5-methyl-5-(1,3,4-oxadiazol-2-yl)-4,5-dihydroisoxazol-3-yl]phenoxy}ethyl)morpholine (compound no. 13),
2-{2-methoxy-5-[5-methyl-5-(1,3,4-oxadiazol-2-yl)-4,5-dihydroisoxazol-3-yl]phenoxy}acetamide (compound no. 14),
and their pharmaceutically acceptable salts, pharmaceutically acceptable solvates, enantiomers, diastereomers or N-oxides.

3. A pharmaceutical composition comprising a therapeutically effective amount of a compound of claim 1 or 2, together with at least one pharmaceutically acceptable carrier, excipient or diluent.

4. A pharmaceutical composition comprising a therapeutically effective amount of a compound of claim 1 or 2 and at least one other active ingredient selected from corticosteroids, =2-agonist, leukotriene antagonists, 5-lipoxygenase inhibitors, chemokine inhibitors, muscarinic receptor antagonists, p38 MAP kinase inhibitors, anticholinergics, antiallergics, PAF antagonists, EGFR kinase inhibitors, additional PDE-IV inhibitors, kinase inhibitors or combinations thereof.

5. A method for treating, preventing, inhibiting or suppressing an inflammatory condition or disease or CNS diseases, in a patient, comprising administering to the said patient a therapeutically effective amount of a compound of claim 1 or 2.

6. A method for treating, preventing, inhibiting or suppressing an inflammatory condition or disease or CNS diseases, in a patient, comprising administering to the said patient a therapeutically effective amount of a pharmaceutical composition of claim 3 or 4.

7. A method for the treatment, prevention, inhibition or suppression of CNS diseases, AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases in a patient comprising administering to said patient a therapeutically effective amount of a compound of claim 1 or 2.

8. A method for the treatment, prevention, inhibition or suppression of CNS diseases, AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases in a patient comprising administering to said patient a therapeutically effective amount of a pharmaceutical composition of claim 3 or 4.

9. A method for the preparation of a compound of Formula IX 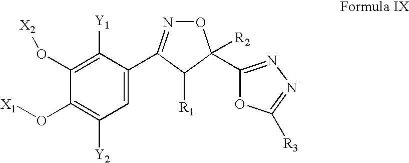
their pharmaceutically acceptable salts, pharmaceutically acceptable solvates, enantiomers, diastereomers or N-oxides, the method comprising:
reacting a compound of Formula II with a compound of Formula X₁Z (wherein Z is halogen) to give a compound of Formula III [wherein X₁ (except hydrogen), Y₁ and Y₂ are the same as defined in claim 1], 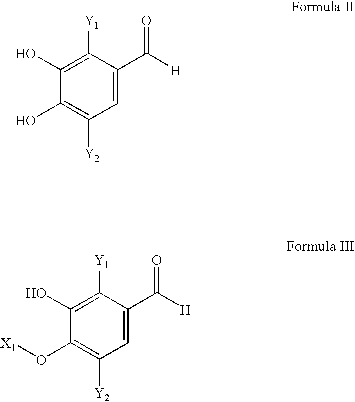
reacting the compound of Formula III with a compound of Formula X₂Z [wherein Z is halogen] to give a compound of Formula IV [wherein X₂ (except hydrogen) is same as defined in claim 1], 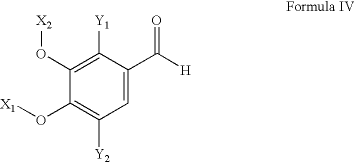
reacting the compound of Formula IV with hydroxylamine hydrochloride to give a compound of Formula V, 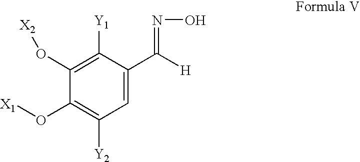
treating the compound of Formula V with a compound of Formula VI to give a compound of Formula VII [wherein R₁ and R₂ are the same as defined in claim 1 and Rr represents COOH, COOCH₃], 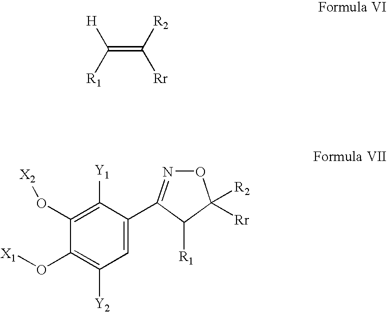
reacting the compound of Formula VII (when Rr is COOCH₃) with hydrazine hydrate to give a compound of Formula VIII, 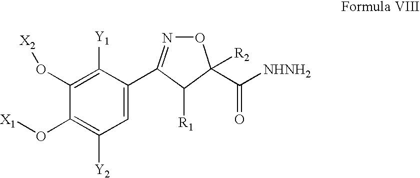
reacting the compound of Formula VIII with a compound of Formula HC(OR₃)₃ to give the compound of Formula IX [wherein R₃ is the same as defined in claim 1],
or debenzylating a compound of Formula X to give a compound of Formula XI [wherein X₁, Y₁, Y₂, R₁, R₂ and R₃ are the same as defined in claim 1], 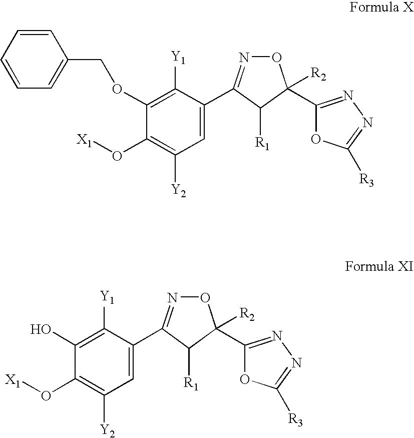
reacting the compound of Formula XI with X₂Z [wherein Z is halogen] to give the compound of Formula IX [wherein X₂ (except hydrogen and benzyl) is same as defined in claim 1].

10. A method for the preparation of a compound of Formula XIII 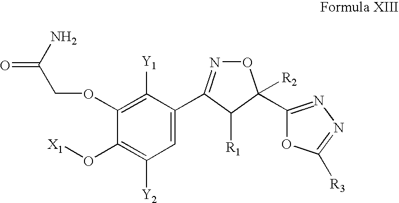
their pharmaceutically acceptable salts, pharmaceutically acceptable solvates, enantiomers, diastereomers or N-oxides, the method comprising:
amidating a compound of Formula XII 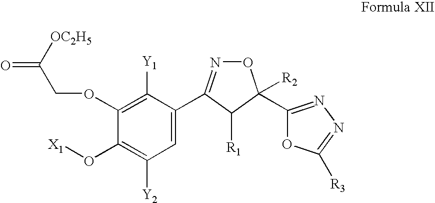
to give the compound of Formula XIII [wherein X₁, Y₁, Y₂, R₁, R₂ and R₃ are the same as defined in claim 1].

11. A method for the preparation of compounds of Formula XVI and Formula XVII 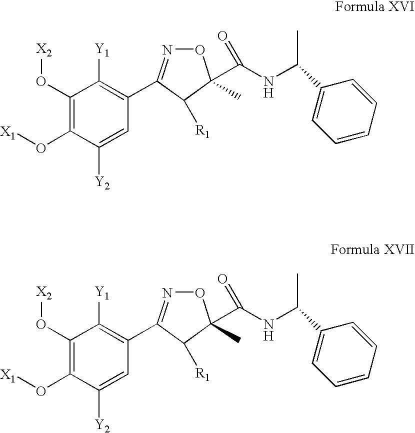
their pharmaceutically acceptable salts, pharmaceutically acceptable solvates, enantiomers, diastereomers or N-oxides, the method comprising:
reacting a compound of Formula XIV with a compound of Formula XV 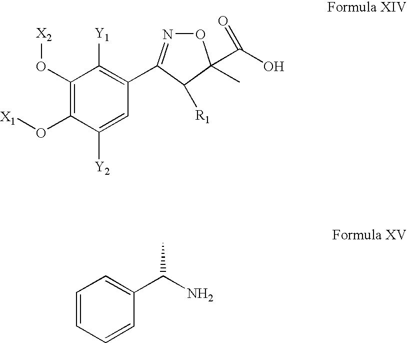
to give the compound of Formula XVI [wherein X₁, X₂, Y₁, Y₂ and R₁ are the same as defined in claim 1] and the compound of Formula XVII [wherein X₁, X₂, Y₁, Y₂ and R₁ are the same as defined in claim 1].

12. A method for the preparation of a compound of Formula XXIII 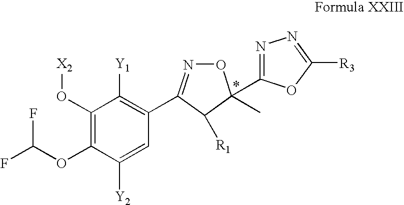
their pharmaceutically acceptable salts, pharmaceutically acceptable solvates, enantiomers, diastereomers or N-oxides, the method comprising:
reacting a compound of Formula XVIII [wherein configuration at stereogenic carbons marked * is (R) or (S)] with hydrazine hydrate to give a compound of Formula XIX, 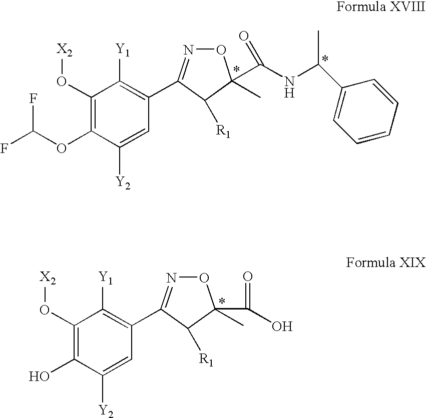
reacting the compound of Formula XIX with methanol to give a compound of Formula XX, 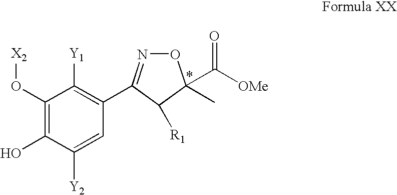
reacting the compound of Formula XX with Freon gas to give a compound of Formula XXI, 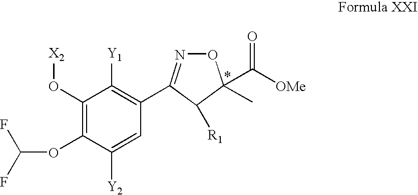
reacting the compound of Formula XXI with hydrazine hydrate to give a compound of Formula XXII, 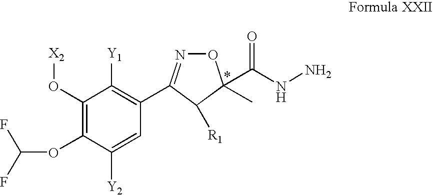
reacting the compound of Formula XXII with a compound of Formula HC(OR₃)₃ to give the compound of Formula XXIII [wherein X₂, Y₁, Y₂, R₁ and R₃ are the same as defined in claim 1 and configuration at stereogenic carbon marked * is (R) or (S)].
